(19) United States
(12) Patent Application Publication
(10) Pub. No.: US 2010/0015042 A1
(43) Pub. Date: Jan. 21, 2010

(54) Combine radiation therapy and chemotherapy for treating cancer

(75) Inventors: Yona Keisari, Ramat Gan (IL); Itzhak Kelson, Tel-Aviv (IL); Tomer Cooks, Givataim (IL)

Correspondence Address: MARTIN D. MOYNIHAN d/b/a PRTSI, INC., P.O. BOX 16446, ARLINGTON, VA, 22215, US

(73) Assignee: Ramot At Tel Aviv University Ltd., Tel-Aviv (IL)

(21) Appl. No.: 12/458,164

(22) Filed: Jul. 2, 2009

Related U.S. Application Data
(60) Provisional application No. 61/129,547, filed on Jul. 3, 2008.

(51) Int. Cl. A61K 51/00; A61P 35/00

(52) U.S. Cl. 424/1.11

(57) ABSTRACT

A method of treating a tumor of a subject is disclosed. The method comprises administering to the subject a therapeutically effective amount of alpha particles and a chemotherapeutic agent, wherein the alpha particles are administered by positioning a non-stable alpha-emitting radionuclide in proximity to and/or within the tumor, so as to administer a dose of alpha particles into the tumor, wherein the method does not comprise administration of an inhibitor of DNA repair, thereby treating the tumor of the subject.

### RELATED APPLICATIONS

This Application claims the benefit of U.S. Provisional Patent Application No. 61/129,547, Filed on Jul. 3, 2008, the contents of which are incorporated herein by reference.

### FIELD AND BACKGROUND OF THE INVENTION

The present invention relates, in some embodiments thereof, to treating cancer, and particularly, but not necessarily, to combined treatment of chemotherapy and radiation therapy.

Cancer is a major cause of death in the modern world. Effective treatment of cancer is most readily accomplished following early detection of malignant tumors. Most techniques used to treat cancer (other than chemotherapy) are directed against a defined tumor site in an organ, such as brain, breast, ovary, colon and the like.

Known in the art are several procedures for treating tumors by irradiation. One such procedure employs laser light, which can destruct unwanted cells either through a direct interaction between the laser beam and the tissue, or through activation of some photochemical reactions using light-activated molecules which are injected into or otherwise administered to the tissue. For example, in a procedure, known as Photo-dynamic therapy (PDT), a photosensitive drug that binds to rapidly dividing cells is administered to the subject. Subsequently, the photosensitive drug is irradiated using a narrow-band laser so as to induce a chemical reaction resulting in a production of reactive products which then destroy the abnormal tissue.

However, most photosensitive agents are activated at wavelengths that can only penetrate through three or less centimeters of tissue. Hence, non- or minimal-invasive PDT can be used for cancerous growths that are on or near the surface of the skin, or on the lining of internal organs.

Radiation therapy, also referred to as radiotherapy, or therapeutic radiology, is the use of radiation sources in the treatment or relief of diseases. Radiotherapy typically makes use of ionizing radiation, deep tissue-penetrating rays, which can physically and chemically react with diseased cells to destroy them. Each therapy program has a radiation dosage defined by the type and amount of radiation for each treatment session, frequency of treatment session and total of number of sessions.

Radiotherapy is particularly suitable for treating solid tumors, which have a well-defined spatial contour. Such tumors are encountered in breast, kidney and prostate cancer, as well as in secondary growths in the brain, lungs and liver.

It is well known that different types of radiation differ widely in their cell killing efficiency. Gamma and beta rays have a relatively low efficiency.

The combination of low-linear energy transfer (LET) (x-rays, gamma rays) radiation therapy (RT) and platinum derivatives is a common anticancer strategy and achieves a better antitumor effect compared with each modality, alone. For example, cisplatin (CP) (described as an apoptosis enhancer that cross-links cellular DNA, forming bifunctional adducts with the N7 of guanine bases) is effective when combined with LET RT in several different malignancies, including both small cell and nonsmall cell lung carcinoma, lymphoma, and head and neck carcinomas [Scagliotti G. J Thorac Oncol. 2007;2 (suppl 2):S86-S91; Mey U J, et al. Cancer Invest. 2006;24:593-600; Colevas A D. J Clin Oncol. 2006;24:2644-2652].

In contrast to x-rays and gamma rays, alpha particles as well as other heavy charged particles are capable of transferring larger amount of energies, hence being extremely efficient. In certain conditions, the energy transferred by a single heavy particle is sufficient to destroy a cell. Moreover, the non-specific irradiation of normal tissue around the target cell is greatly reduced or absent because heavy particles can deliver the radiation over the distance of a few cells diameters. On the other hand, the fact that their range in human tissue is less than 0.1 millimeter, limits the number of procedures in which heavy particles can be used. More specifically, conventional radiotherapy by alpha particles is typically performed externally when the tumor is on the surface of the skin.

U.S. Patent Application Publication No. 20070041900 to Kelson et al. teaches an intra-tumoral radiotherapy method with alpha particles.

Cooks et al [Cancer, Apr. 15, 2009] teaches the effect of a combination therapy comprising a chemotherapeutic agent and radiotherapy with alpha particles.

U.S. Patent Application 20040018968 teaches histone deacetylase inhibitors (agents which inhibit DNA repair) in combination with radiation for the treatment of cancer.

U.S. Patent Application 20050222013 teaches histone deacetylase inhibitors in combination with radiation for the treatment of cancer. The histone deacetylase inhibitor may be administered together with additional chemotherapeutic agents such as cisplatin.

U.S. Pat. No. 6,391,911 teaches co-administration of lucanthone (an agent which inhibits excision repair of damage induced by radiation) and radiation for treatment of cancer.

U.S. Pat. No. 6,392,068 teaches delivery of a non-active (or stable) radioisotope which following exposure to neutrons emits alpha particles for the treatment of cancer.

### SUMMARY OF THE INVENTION

According to an aspect of some embodiments of the present invention there is provided a method of treating a tumor of a subject, the method comprising administering to the subject a therapeutically effective amount of alpha particles and a chemotherapeutic agent, wherein the alpha particles are administered by positioning a non-stable alpha-emitting radionuclide in proximity to and/or within the tumor, so as to administer a dose of alpha particles into the tumor, wherein the method does not comprise administration of an inhibitor of DNA repair, thereby treating the tumor of the subject.

According to an aspect of some embodiments of the present invention there is provided a method of treating a tumor of a subject, the method comprising administering to the subject a therapeutically effective amount of alpha particles and a chemotherapeutic agent, wherein the chemotherapeutic agent is administered systemically, wherein the alpha particles are administered by positioning a non-stable alpha-emitting radionuclide in proximity to and/or within the tumor, so as to administer a dose of alpha particles into the tumor and wherein the chemotherapeutic agent is selected from the group consisting of cisplatin, gemcitabine, 5-fluorouracil (5FU), taxol and doxorubicin, thereby treating the tumor of the subject.

According to some embodiments of the invention, the tumor is a solid tumor.

According to some embodiments of the invention, the non-stable alpha-emitting radionuclide is selected from the group consisting of Radium-223, Radium-224, Radon-219 and Radon-220.

According to some embodiments of the invention, the positioning of the non-stable alpha-emitting radionuclide is effected by at least one radiotherapy device having a surface whereby the alpha-emitting radionuclide is on or beneath the surface.

According to some embodiments of the invention, the at least one radiotherapy device comprises a wire.

According to some embodiments of the invention, the non-stable alpha-emitting radionuclide is comprised in a solution.

According to some embodiments of the invention, the positioning is effected at the base of the tumor.

According to some embodiments of the invention, the at least one radiotherapy device comprises two radiotherapy devices.

According to some embodiments of the invention, the tumor is selected from the group consisting of a squamous cell carcinoma tumor (SCC tumor), a pancreatic carcinoma tumor and a colon carcinoma tumor.

According to some embodiments of the invention, the chemotherapeutic agent is selected from the group consisting of cisplatin, gemcitabine, is 5-fluorouracil (5FU), taxol and doxorubicin.

According to some embodiments of the invention, when the tumor is a SCC tumor, the chemotherapeutic agent is cisplatin.

According to some embodiments of the invention, when the tumor is a pancreatic carcinoma tumor, the chemotherapeutic agent is gemcitabine.

According to some embodiments of the invention, when the tumor is a colon carcinoma tumor, the chemotherapeutic agent is 5-fluorouracil (5FU).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are graphs showing the inhibition effect of combined diffusing alpha-emitter radiation therapy (DART)/chemotherapy on cell proliferation 48 hours (FIG. 1A) and 72 hours (FIG. 1B) following treatment.

FIGS. 2A-F are graphs showing apoptosis induction by combined DART/chemotherapy as measured by flow cytometry. FIG. 2A: untreated cells (control); FIG. 2B—cells exposed to 0.8 Gy of alpha particles; FIG. 2C—cells exposed to 2.4 Gy of alpha particles; FIG. 2D cells treated with 30 μM cisplatin for 4 hours; FIG. 2E—cells treated with both 0.8 Gy of alpha particles and 30 μM cisplatin for 4 hours; FIG. 2F—cells treated with both 2.4 Gy of alpha particles and 30 μM cisplatin for 4 hours.

FIG. 2G is a graph showing percentage of apoptotic cells found at the same groups, as analyzed by flow cytometry.

FIG. 3 is a graph showing squamous cell tumor growth inhibition by chemotherapy, DART therapy, and DART/chemotherapy combination according to an embodiment of the invention. In the legend: Inert−Tumor bearing mice treated with inert wires (n=15); Inert+CP−Tumor bearing mice treated with inert wires and cisplatin (n=15); ²²⁴Ra wire−tumor bearing mice treated each with one radioactive wire loaded with ²²⁴Ra atoms (n=14); ²²⁴Ra wire+CP−Tumor bearing mice treated with one radioactive wire loaded with ²²⁴Ra atoms and cisplatin (n=15).

FIGS. 4A-B are graphs showing tumor growth inhibition (FIG. 4A) and prolonged survival (FIG. 4B) following cisplatin combined with a double 224Ra wire insertion. BALB/c mice bearing SQ2 tumors, were treated with either two Ra-224 wires or by two separate doses of cisplatin (5 mg/kg each) or both, and monitored for tumor growth and survival. In the legends: Inert−Tumor bearing mice treated with inert wires (n=15). Inert+CP−Tumor bearing mice treated with inert wires and cisplain (n=15). ²²⁴Ra wire−Tumor bearing mice treated with radioactive wires loaded with ²²⁴Ra atoms (n=14). ²²⁴Ra wire+CP−Tumor bearing mice treated with radioactive wires loaded with ²²⁴Ra atoms and cisplatin (n=15).

FIGS. 5A-B are photographs of hematoxylin-eosin (H&E) stained cross lung sections (×10 magnitude) from mice having received DART/chemotherapy according to an embodiment of the invention (FIG. 5B) and control mice (FIG. 5A).

FIG. 5C is a bar graph showing the ratio between lung of mice treated with inert wires compared to those treated with both cisplatin and DART (together and alone) in respect of normal healthy lungs of mice with no tumors.

FIG. 6 is a graph showing tumor growth retardation by a single ²²⁴Ra wire combined with Gemzar (60 mg/kg) compared to ²²⁴Ra wire group, inert wire group and Gemzar+inert wire group. Initial tumor size 4.93 mm length±0.12 (STE).

FIG. 7 is a graph showing the effect of two ²²⁴Ra-loaded wires combined with 5-FU treatment on colon cancers. Treatment was applied to Balb/c mice bearing 6-7 mm in diameter tumors. Two ²²⁴Ra wires: Tumor bearing mice treated with 2 ²²⁴Ra wires, carrying activities in the range of 27.9-35.5 kBq (n=5). Two ²²⁴Ra wires combined with 5-FU: administration of 75 mg/kg 5-FU 24 hours prior to treatment with 2 ²²⁴Ra wires, carrying activities in the range of 32.1-33.8 kBq (n=5). Two Inert wires combined with 5-FU: administration of 75 mg/kg 5-FU 24 hours prior to treatment with 2 inert wires (n=6). Two Inert wires: Tumor bearing mice treated with 2 inert wires (n=6).

### DESCRIPTION OF SPECIFIC EMBODIMENTS OF THE INVENTION

The present invention relates, in some embodiments thereof, to treating cancer, and particularly, but not necessarily, to combined treatment of chemotherapy and radiation therapy.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

It is well known that different types of radiation differ widely in their cell killing efficiency. Gamma and beta rays have a relatively low efficiency, whilst alpha particles as well as other heavy charged particles are capable of transferring larger amount of energies, hence being extremely efficient. The low efficiency of gamma and beta rays has necessitated the search for combination therapies, whereby cancer patients are treated both with radiation and chemotherapeutic agents. Due to the high efficiency of alpha particles, it has never been suggested to combine such radiotherapy with chemotherapy except in the case of agents that prevent DNA repair following radiation induced DNA damage (i.e. radiation sensitizing agents).

The present inventors surprisingly found that the lethal effect of intratumoral administration of alpha emitting particles on cancer cells could be enhanced by chemotherapeutic agents such as cisplatin, gemcitabine and 5-fluorouracil.

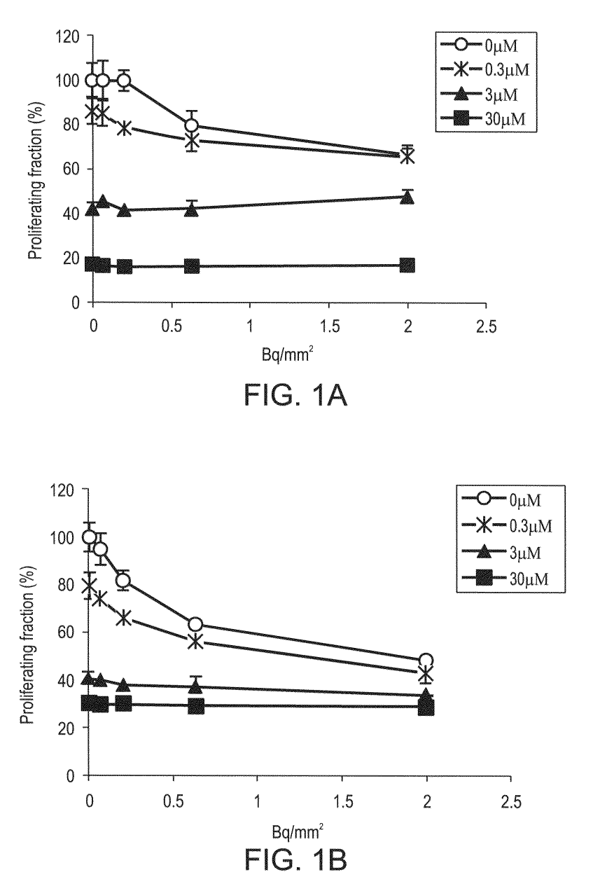

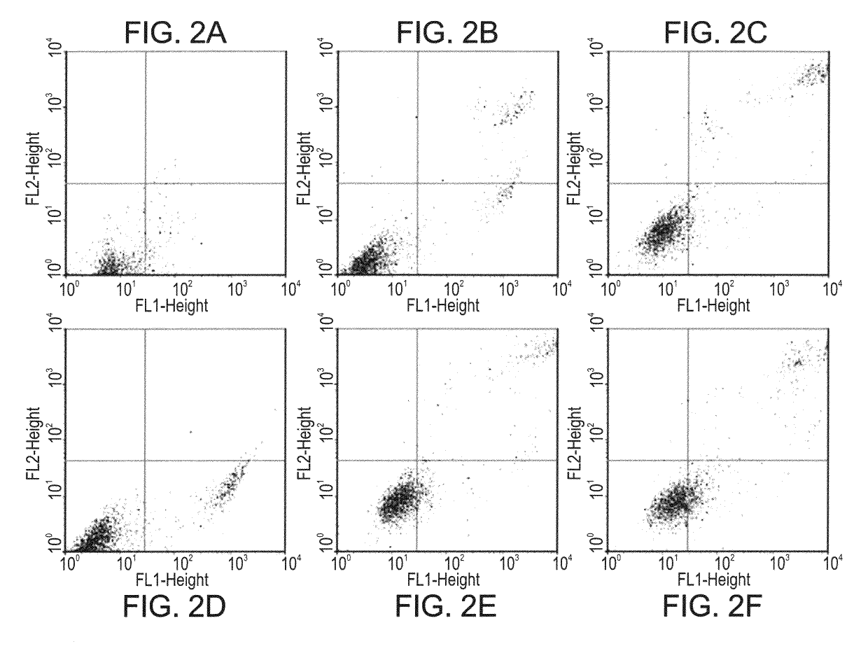

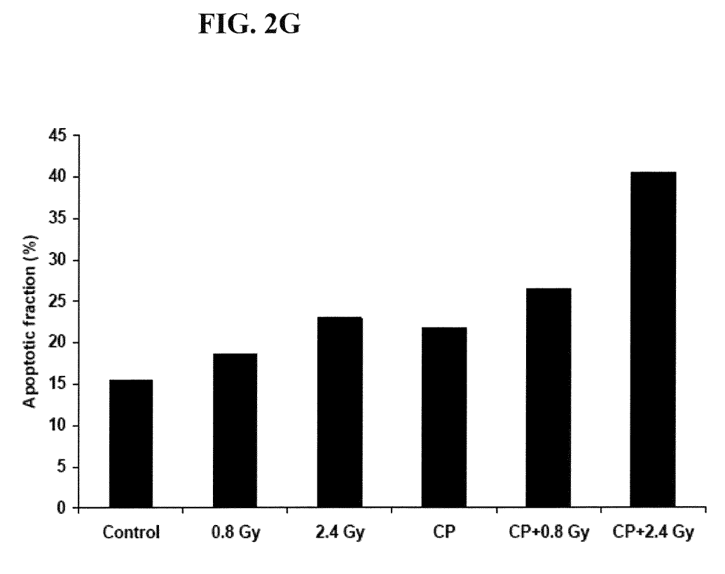

Whilst reducing the present invention to practice, the present inventors found that the combination of alpha particles and cisplatin decreased proliferation of cancer cells (SQ2 cells) in vitro to a greater extent than either treatment alone (FIGS. 1A-B). In addition, the combination of alpha particles and cisplatin decreased apoptosis of cancer cells in vitro to a greater extent than either treatment alone (FIG. 2G).

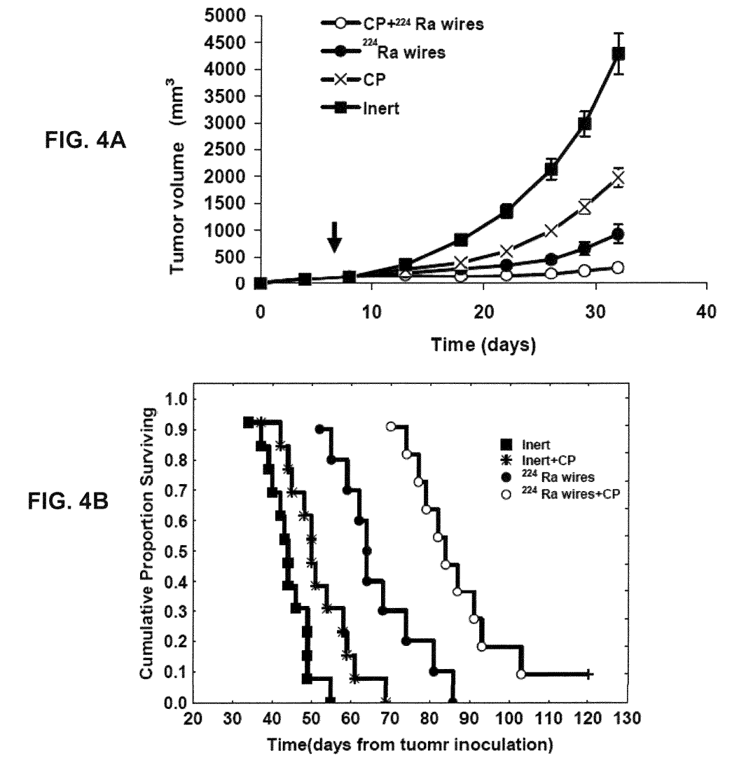

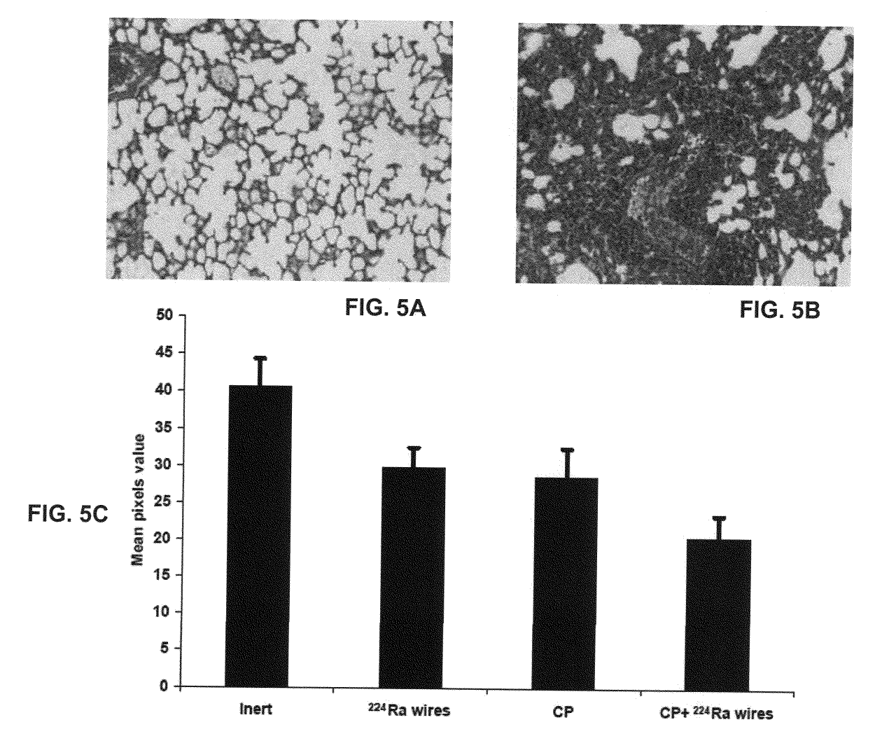

In vivo data suggests that there is a synergistic effect between alpha radiation and cisplatin. Thus, the survival prolongation of the combined therapy was much higher than the sum of prolongation achieved with each therapy alone (FIG. 4B). The higher efficiency of the combined treatment was also confirmed by histological examination (FIGS. 5A-C).

Whilst further reducing the invention to practice, the present inventors showed that the combination of alpha particles and a chemotherapeutic agent was beneficial for the treatment of cancers other than lung cancers such as pancreatic carcinomas and colon carcinomas. Further, the present inventors demonstrated the beneficial effect of using combined therapy with alpha particle radiation using two additional chemotherapeutic agents—gemcitabine and 5-fluorouracil.

It will be appreciated that such synergistic activity of alpha radiation treatment with additional chemotherapeutic compositions has the potential to significantly reduce the effective clinical doses of such treatments, thereby reducing the often devastating negative side effects and high cost of the treatment.

Thus, according to one aspect of the present invention there is provided a method of treating a solid tumor of a subject, the method comprising administering to the subject a therapeutically effective amount of alpha particles and a chemotherapeutic agent, wherein the alpha particles are administered by positioning a non-stable alpha-emitting radionuclide in proximity to and/or within the tumor, so as to administer a dose of alpha particles into the solid tumor, wherein the method does not comprise administration of an inhibitor of DNA repair, thereby treating the solid tumor of the subject.

The term “tumor” as used herein, refers to an abnormal mass of tissue including benign and malignant cancers. Exemplary tumors (including both solid tumor and non-solid tumors) and tumoral related diseases that can be treated according to this method of the present invention include tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic ; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypemephroma, insulinoma, islet tumor, keratoma, leiomyoblastoma, leiomyosarcoma, lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

As used herein “in proximity to a tumor” refers to a sufficient distance for allowing alpha particles or decay chain nuclei of the radionuclide to arrive at the tumor. Preferably, the distance between the radionuclide and the tumor is less than 0.1 mm, more preferably less than 0.05 mm, most preferably less than 0.001 mm.

According to a preferred embodiment of the present invention, the amount of radionuclide and the time of exposure are selected such that there is sufficient time to administer a predetermined therapeutic dose of decay chain nuclei and alpha particles into the tumor.

The non-stable radionuclide is preferably a relatively short lived radio-isotope, such as, but not limited to, Radium-223, Radium-224, Radon-219, Radon-220 and the like. Accordingly, the present invention does not envisage the use of boronated compounds such as described in U.S. Pat. No. 6,392,068 which are stable and only upon exposure to neutrons do they emit radiation.

When Radium 223 is employed, the following decay chain is emitted therefrom:

Ra-223 decays, with a half-life period of 11.4 d, to Rn-219 by alpha emission;

Rn-219 decays, with a half-life period of 4 s, to Po-215 by alpha emission;

Po-215 decays, with a half-life period of 1.8 ms, to Pb-211 by alpha emission;

Pb-211 decays, with a half-life period of 36 m, to Bi-211 by beta emission;

Bi-211 decays, with a half-life period of 2.1 m, to Tl-207 by alpha emission; and

Tl-207 decays, with a half-life period of 4.8 m, to stable Pb-207 by beta emission.

As can be understood from the above decay chain, when Rn-219 is employed as the radionuclide, the decay chain begins with the decay of Rn-219 to Po-215, and continues to Pb-211, Bi-211, Tl-207 and Pb-207.

When Radium 224 is employed, the following decay chain is emitted therefrom:

Ra-224 decays, with a half-life period of 3.7 d, to Rn-220 by alpha emission;

Rn-220 decays, with a half-life period of 56 s, to Po-216 by alpha emission;

Po-216 decays, with a half-life period of 0.15 s, to Pb-212 by alpha emission;

Pb-212 decays, with a half-life period of 10.6 h, to Bi-212 by beta emission;

Bi-212 decays, with a half-life of 1 h, to Tl-208 by alpha emission (36% branching ratio), or to Po-212 by beta emission (64% branching ratio);

Tl-208 decays, with a half-life of 3 m, to stable Pb-208 by beta emission; and

Po-212 decays, with a half-life of 0.3 μs, to stable Pb-208 by alpha emission.

As can be understood from the above decay chain, when Rn-220 is employed as the radionuclide, the decay chain begins with the decay of Rn-220 to Po-216, and continues to Pb-212, Bi-212, Tl-208 (or Po-212) and Pb-208.

In any event when the radionuclide is positioned in proximity to and/or within a tumor, a plurality of short-lived atoms are released into the surrounding environment and dispersed therein by thermal diffusion and/or by convection via body fluids. The short-lived atoms and their massive decay products (i.e., alpha particles and daughters nuclei), either interact with the cells of the tumor or continue the decay chain by producing smaller mass particles. As will be appreciated by one ordinarily skilled in the art, the close proximity between the radionuclide and the tumor, and the large number of particles which are produced in each chain, significantly increase the probability of damaging the cells of interest, hence allowing for an efficient treatment of the tumor.

Methods of administering alpha particles to tumors and devices for same are known in the art—see for example U.S. Pat. Application No. 20070041900, incorporated herein by reference.

According to one embodiment the alpha particles are administered to the tumor using a radiotherapy device having a surface whereby the alpha-emitting radionuclide is on or beneath the surface (e.g. a wire).

Typically, the non-stable alpha-emitting radionuclide is comprised in a solution. The wire is typically dipped into the solution as described in the Materials and Methods of the Examples section herein below.

The alpha emitting radionuclide may be administered at any position of the tumor. According to a preferred embodiment, the radionuclide is administered at the base of the tumor.

The present invention contemplates concomitant administration of more than one device—e.g. two radiotherapy devices. The devices may be loaded with an identical or non-identical alpha emitting radionuclide. The devices may be loaded at the same positions on the tumor—e.g. both at the base of the tumor. Alternatively, the devices may be loaded at non-identical positions—e.g. one at the base and one at the tip of the tumor.

As mentioned, the method of the present invention is effected by co-administering alpha emitting radionuclides with a chemotherapeutic agent.

As used herein, the phrase “chemotherapeutic agent” refers to an agent (e.g. chemical agent, polypeptide agent, polynucleotide agent etc.), which is capable of inhibiting, disrupting, preventing or interfering with cell growth and/or proliferation, without the need of an additional agent. Examples of chemotherapeutic agents include, but are not limited to, agents which induce apoptosis, necrosis, mitotic cell death, alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, steroid hormones and anti-androgens.

According to one embodiment, the chemotherapeutic agent is not an agent which only inhibits DNA repair (e.g. histone deacetylase inhibitors or lucanthone). According to another embodiment only one single chemotherapeutic agent is administered. Alternatively, more than one chemotherapeutic agent may be administered, but with the proviso that the chemotherapeutic agent is not an agent which only inhibits DNA repair.

Exemplary chemotherapeutic agents and uses thereof are provided in Table 1 herein below.

According to one embodiment, the chemotherapeutic agent is selected from the group consisting of cisplatin, gemcitabine, is 5-fluorouracil (5FU), taxol and doxorubicin.

TABLE 1

| | | | Manufacturer/ |
|---|---|---|---|
| Drug | Drug Trade Name | Approved Use | Distributor |
| abarelix | Plenaxis depot | For the palliative treatment of men | Praecis |
| | | with advanced symptomatic | |
| | | prostate cancer, in whom LHRH | |
| | | agonist therapy is not appropriate | |
| | | and who refuse surgical castration, | |
| | | and have one or more of the | |
| | | following: (1) risk of neurological | |
| | | compromise due to metastases, (2) | |
| | | ureteral or bladder outlet | |
| | | obstruction due to local | |
| | | encroachment or metastatic | |
| | | disease, or (3) severe bone pain | |
| | | from skeletal metastases persisting | |
| | | on narcotic analgesia | |
| aldesleukin | Prokine | Treatment of adults with metastatic | Chiron |
| | | melanoma | |
| Aldesleukin | Proleukin | Treatment of adults with metastatic | Chiron Corp |
| | | renal cell carcinoma | |
| Alemtuzumab | Campath | Accel. Approv. (clinical benefit not | Millennium and |
| | | established) Campath is indicated | ILEX Partners, LP |
| | | for the treatment of B-cell chronic | |
| | | lymphocytic leukemia (B-CLL) in | |
| | | patients who have been treated | |
| | | with alkylating agents and who | |
| | | have failed fludarabine therapy. | |
| alitretinoin | Panretin | Topical treatment of cutaneous | Ligand |
| | | lesions in patients with AIDS- | Pharmaceuticals |
| | | related Kaposi's sarcoma. | |
| allopurinol | Zyloprim | Patients with leukemia, lymphoma | GlaxoSmithKline |
| | | and solid tumor malignancies who | |
| | | are receiving cancer therapy which | |
| | | causes elevations of serum and | |
| | | urinary uric acid levels and who | |
| | | cannot tolerate oral therapy. | |
| altretamine | Hexalen | Single agent palliative treatment of | US Bioscience |
| | | patients with persistent or recurrent | |
| | | ovarian cancer following first-line | |
| | | therapy with a cisplatin and/or | |
| | | alkylating agent based | |
| | | combination. | |
| amifostine | Ethyol | To reduce the cumulative renal | US Bioscience |
| | | toxicity associated with repeated | |
| | | administration of cisplatin in | |
| | | patients with advanced ovarian | |
| | | cancer | |
| amifostine | Ethyol | Accel. Approv. (clinical benefit not | US Bioscience |
| | | established) Reduction of platinum | |
| | | toxicity in non-small cell lung | |
| | | cancer | |
| amifostine | Ethyol | To reduce post-radiation | US Bioscience |
| | | xerostomia for head and neck | |
| | | cancer where the radiation port | |
| | | includes a substatial portion of the | |
| | | parotid glands. | |
| anastrozole | Arimidex | Accel. Approv. (clinical benefit not | AstraZeneca |
| | | established) for the adjuvant | |
| | | treatment of postmenopausal | |
| | | women with hormone receptor | |
| | | positive early breast cancer | |
| anastrozole | Arimidex | Conversion to regular approval for | AstraZeneca |
| | | the adjuvant treatment of | |
| | | postmenopausal women with | |
| | | hormone receptor positive early | |
| | | breast cancer | |
| anastrozole | Arimidex | Treatment of advanced breast | AstraZeneca |
| | | cancer in postmenopausal women | Pharmaceuticals |
| | | with disease progression following | |
| | | tamoxifen therapy. | |
| anastrozole | Arimidex | For first-line treatment of | AstraZeneca |
| | | postmenopausal women with | Pharmaceuticals |
| | | hormone receptor positive or | |
| | | hormone receptor unknown locally | |
| | | advanced or metastatic breast | |
| | | cancer. | |
| arsenic trioxide | Trisenox | Second line treatment of relapsed | Cell Therapeutic |
| | | or refractory APL following ATRA | |
| | | plus an anthracycline. | |
| asparaginase | Elspar | Therapy of patients with acute | Merck |
| | | lymphocytic leukemia | |
| Asparaginase | Elspar | ELSPAR is indicated in the therapy | Merck & Co, Inc |
| | | of patients with acute lymphocytic | |
| | | leukemia. This agent is useful | |
| | | primarily in combination with | |
| | | other chemotherapeutic agents in | |
| | | the induction of remissions of the | |
| | | disease in pediatric patients. | |
| azacitidine | Vidaza | For use for the treatment of | Pharmion |
| | | patients with the following | |
| | | myelodysplastic syndrome | |
| | | subtypes: refractory anemia or | |
| | | refractory anemia with ringed | |
| | | sideroblasts (if accompanied by | |
| | | neutropenia or thrombocytopenia | |
| | | and requiring transfusions), | |
| | | refractory anemia with excess | |
| | | blasts, refractory anemia with | |
| | | excess blasts in transformation, and | |
| | | chronic myelomonocytic leukemia | |
| bevacuzimab | Avastin | First-line treatment of patients with | Genentech |
| | | metastatic carcinoma of the colon | |
| | | and rectum (in combination with | |
| | | intravenous 5-fluorouracil-based | |
| | | chemotherapy) | |
| bexarotene capsules | Targretin | For the treatment by oral capsule of | Ligand Pharmaceuticals |
| | | cutaneous manifestations of | |
| | | cutaneous T-cell lymphoma in | |
| | | patients who are refractory to at | |
| | | least one prior systemic therapy. | |
| bexarotene gel | Targretin | For the topical treatment of | Ligand Pharmaceuticals |
| | | cutaneous manifestations of | |
| | | cutaneous T-cell lymphoma in | |
| | | patients who are refractory to at | |
| | | least one prior systemic therapy. | |
| bleomycin | Blenoxane | | Bristol-Myers Squibb |
| bleomycin | Blenoxane | Sclerosing agent for the treatment | Bristol-Myers Squibb |
| | | of malignant pleural effusion | |
| | | (MPE) and prevention of recurrent | |
| | | pleural effusions. | |
| bortezomib | Velcade | Accel. Approv. (clinical benefit not | Millenium |
| | | established) for the treatment of | |
| | | multiple myeloma patients who | |
| | | have received at least two prior | |
| | | therapies and have demonstrated | |
| | | disease progression on the last | |
| | | therapy | |
| bortezomib | Velcade | Conversion to regular approval for | Millenium |
| | | treatment of multiple myeloma | |
| | | patients who have received as least | |
| | | one prior therapy | |
| busulfan intravenous | Busulfex | Use in combination with | Orphan Medical, Inc |
| | | cyclophoshamide as conditioning | |
| | | regimen prior to allogeneic | |
| | | hematopoietic progenitor cell | |
| | | transplantation for chronic | |
| | | myelogenous leukemia. | |
| busulfan oral | Myleran | Chronic Myelogenous Leukemia- | GlaxoSmithKline |
| | | palliative therapy | |
| calustcrone | Methosarb | | Pharmacia & Upjohn |
| | | | Company |
| capecitabine | Xeloda | Accel. Approv. (clinical benefit | Roche |
| | | subsequently established) | |
| | | Treatment of metastatic breast | |
| | | cancer resistant to both paclitaxel | |
| | | and an anthracycline containing | |
| | | chemotherapy regimen or resistant | |
| | | to paclitaxel and for whom further | |
| | | anthracycline therapy may be | |
| | | contraindicated, e.g., patients who | |
| | | have received cumulative doses of | |
| | | 400 mg/m2 of doxorubicin or | |
| | | doxorubicin equivalents | |
| capecitabine | Xeloda | Initial therapy of patients with | Roche |
| | | metastatic colorectal carcinoma | |
| | | when treatment with | |
| | | fluoropyrimidine therapy alone is | |
| | | preferred. Combination | |
| | | chemotherapy has shown a survival | |
| | | benefit compared to 5-FU/LV | |
| | | alone. A survival benefit over | |
| | | 5_FU/LV has not been | |
| | | demonstrated with Xeloda | |
| | | monotherapy. | |
| capecitabine | Xeloda | Conversion to regular approval for | Roche |
| | | treatment in combination with | |
| | | docetaxel of patients with | |
| | | metastatic breast cancer after | |
| | | failure of prior anthracycline | |
| | | containing chemotherapy | |
| capecitabine | Xeloda | Adjuvant treatment in patients with | Roche |
| | | Dukes' C colon cancer who have | |
| | | undergone complete resection of | |
| | | the primary tumor when treatment | |
| | | with fluoropyrimidine therapy | |
| | | alone is preferred | |
| carboplatin | Paraplatin | Palliative treatment of patients with | Bristol-Myers Squibb |
| | | ovarian carcinoma recurrent after | |
| | | prior chemotherapy, including | |
| | | patients who have been previously | |
| | | treated with cisplatin. | |
| carboplatin | Paraplatin | Initial chemotherapy of advanced | Bristol-Myers Squibb |
| | | ovarian carcinoma in combination | |
| | | with other approved | |
| | | chemotherapeutic agents. | |
| carmustine | BCNU, BiCNU | | Bristol-Myers Squibb |
| carmustine | Gliadel | Treatment of patients with | MGI Pharma |
| | | malignant glioma undergoing | |
| | | primary surgical resection | |
| carmustine with | Gliadel Wafer | For use in addition to surgery to | Guilford Pharmaceuticals |
| Polifeprosan 20 | | prolong survival in patients with | Inc. |
| Implant | | recurrent glioblastoma multiforme | |
| | | who qualify for surgery. | |
| cetuximab | Erbitux | Accel. Approv. (clinical benefit not | Imclone |
| | | established) for treatment of | |
| | | EGFR-expressing metastatic | |
| | | colorectal carcinoma in patients | |
| | | who are refractory to irinotecan- | |
| | | based chemotherapy (in | |
| | | combination with irinotecan); as a | |
| | | single agent, treatment of EGFR- | |
| | | expressing metastatic colorectal | |
| | | carcinoma in patients who are | |
| | | intolerant to irinotecan-based | |
| | | chemotherapy | |
| cetuximab | Erbirux | For use in combination with | Imclone |
| | | radiation therapy (RT) for the | |
| | | treatment of locally or regionally | |
| | | advanced squamous cell carcinoma | |
| | | of the head and neck (SCCHN) or | |
| | | as a single agent for the treatment | |
| | | of patients with recurrent or | |
| | | metastatic SCCHN for whom prior | |
| | | platinum-based therapy has failed. | |
| chlorambucil | Leukeran | | GlaxoSmithKline |
| cisplatin | Platinol | Metastatic testicular-in established | Bristol-Myers Squibb |
| | | combination therapy with other | |
| | | approved chemotherapeutic agents | |
| | | in patients with metastatic | |
| | | testicular tumors whoc have | |
| | | already received appropriate | |
| | | surgical and/or radiotherapeutic | |
| | | procedures. An established | |
| | | combination therapy consists of | |
| | | Platinol, Blenoxane and Velbam. | |
| cisplatin | Platinol | Metastatic ovarian tumors-in | Bristol-Myers Squibb |
| | | established combination therapy | |
| | | with other approved | |
| | | chemotherapeutic agents: Ovarian- | |
| | | in established combination therapy | |
| | | with other approved | |
| | | chemotherapeutic agents in patients | |
| | | with metastatic ovarian tumors | |
| | | who have already received | |
| | | appropriate surgical and/or | |
| | | radiotherapeutic procedures. An | |
| | | established combination consists of | |
| | | Platinol and Adriamycin. Platinol, | |
| | | as a single agent, is indicated as | |
| | | secondary therapy in patients with | |
| | | metastatic ovarian tumors | |
| | | refractory to standard | |
| | | chemotherapy who have not | |
| | | previously received Platinol | |
| | | therapy. | |
| cisplatin | Platinol | as a single agent for patients with | Bristol-Myers Squibb |
| | | transitional cell bladder cancer | |
| | | which is no longer amenable to | |
| | | local treatments such as surgery | |
| | | and/or radiotherapy. | |
| cladribine | Leustatin, 2-CdA | Treatment of active hairy cell | R.W. Johnson |
| | | leukemia. | Pharmaceutical Research |
| | | | Institute |
| clofarabine | Clolar | Accel. Approv. (clinical benefit not | Genzyme |
| | | established) for the treatment of | |
| | | pediatric patients 1 to 21 years old | |
| | | with relapsed or refractory acute | |
| | | lymphoblastic leukemia after at | |
| | | least two prior regimens | |
| cyclophosphamide | Cytoxan, Neosar | | Bristol-Myers Squibb |
| cyclophosphamide | Cytoxan Injection | | Bristol-Myers Squibb |
| cyclophosphamide | Cytoxan Injection | | Bristol-Myers Squibb |
| cyclophosphamide | Cytoxan Tablet | | Bristol-Myers Squibb |
| cytarabine | Cytosar-U | | Pharmacia & Upjohn |
| | | | Company |
| cytarabine liposomal | DepoCyt | Accel. Approv. (clinical benefit not | Skye Pharmaceuticals |
| | | established) Intrathecal therapy of | |
| | | lymphomatous meningitis | |
| dacarbazine | DTIC-Dome | | Bayer |
| dactinomycin, | Cosmegen | | Merck |
| actinomycin D | | | |
| dactinomycin, | Cosmegan | | Merck |
| actinomycin D | | | |
| Darbepoetin alfa | Aranesp | Aranesp is indicated for the | Amgen, Inc |
| | | treatment of anemia in patients | |
| | | with non-myeloid malignancies | |
| | | where anemia is due to the effect of | |
| | | concomitantly administered | |
| | | chemotherapy. | |
| daunorubicin | DanuoXome | First line cytotoxic therapy for | Nexstar, Inc. |
| liposomal | | advanced, HIV related Kaposi's | |
| | | sarcoma. | |
| daunorubicin, | Daunorubicin | Leukemia/myelogenous/monocytic/ | Bedford Labs |
| daunomycin | | erythroid of adults/remission | |
| | | induction in acute lymphocytic | |
| | | leukemia of children and adults. | |
| daunorubicin, | Cerubidine | In combination with approved | Wyeth Ayerst |
| daunomycin | | anticancer drugs for induction of | |
| | | remission in adult ALL. | |
| decitabine | Dacogen | for the treatment of patients with | MGI PHARMA INC |
| | | myelodysplastic syndromes (MDS) | |
| | | including previously treated and | |
| | | untreated, de novo and secondary | |
| | | MDS of all French-American- | |
| | | British subtypes (refractory | |
| | | anemia, refractory anemia with | |
| | | ringed sideroblasts, refractory | |
| | | anemia with excess blasts, | |
| | | refractory anemia with excess | |
| | | blasts in transformation, and | |
| | | chronic myelomonocytic leukemia) | |
| | | and intermediate-1, intermediate-2, | |
| | | and high-risk International | |
| | | Prognostic Scoring System groups. | |
| Denileukin diftitox | Ontak | Accel. Approv. (clinical benefit not | Seragen, Inc |
| | | established) treatment of patients | |
| | | with persistent or recurrent | |
| | | cutaneous T-cell lymphoma whose | |
| | | malignant cells express the CD25 | |
| | | component of the IL-2 receptor | |
| dexrazoxane | Zinecard | Accel. Approv. (clinical benefit | Pharmacia & Upjohn |
| | | subsequently established) | Company |
| | | Prevention of cardiomyopathy | |
| | | associated with doxorubicin | |
| | | administration | |
| dexrazoxane | Zinecard | Conversion to regular approval for | Pharmacia & Upjohn |
| | | reducing the incidence and severity | Company |
| | | of cardiomyopathy associated with | |
| | | doxorubicin administration in | |
| | | women with metastatic breast | |
| | | cancer who have received a | |
| | | cumulative doxorubicin dose of | |
| | | 300 mg/m2 and who will continue | |
| | | to receive doxorubicin therapy to | |
| | | maintain tumor control. It is not | |
| | | recommended for use with the | |
| | | initiation of doxorubicin therapy. | |
| docetaxel | Taxotere | Accel. Approv. (clinical benefit | Aventis Pharmaceutical |
| | | subsequently established) | |
| | | Treatment of patients with locally | |
| | | advanced or metastatic breast | |
| | | cancer who have progressed during | |
| | | anthracycline-based therapy or | |
| | | have relapsed during anthracycline- | |
| | | based adjuvant therapy. | |
| docetaxel | Taxotere | Conversion to regular approval — | Aventis Pharmaceutical |
| | | treatment of locally advanced or | |
| | | metastatic breast cancer which has | |
| | | progressed during anthracycline- | |
| | | based treatment or relapsed during | |
| | | anthracycline-based adjuvant | |
| | | therapy. | |
| docetaxel | Taxotere | For locally advanced or metastatic | Aventis Pharmaceutical |
| | | non-small cell lung cancer after | |
| | | failure of prior platinum-based | |
| | | chemotherapy. | |
| docetaxel | Taxotere | for use in combination with | Aventis Pharmaceutical |
| | | cisplatin for the treatment of | |
| | | patients with unresectable, locally | |
| | | advanced or metastatic non-small | |
| | | cell lung cancer who have not | |
| | | previously received chemotherapy | |
| | | for this condition cisplatin for the | |
| | | treatment of patients with | |
| | | unresectable, locally advanced or | |
| | | metastatic non-small cell lung | |
| | | cancer who have not previously | |
| | | received chemotherapy for this | |
| | | condition. | |
| docetaxel | Taxotere | For use in combination with | Aventis Pharmaceutical |
| | | prednisone as a treatment for | |
| | | patients with androgen independent | |
| | | (hormone refractory) metastatic | |
| | | prostate cancer | |
| docetaxel | Taxotere | For use in combination with | Aventis Pharmaceutical |
| | | doxorubicin and cyclophosphamide | |
| | | for the adjuvant treatment of | |
| | | patients with operable nodepositive | |
| | | breast cancer | |
| doxorubicin | Adriamycin PFS | For use in combination with | Pharmacia |
| | | cyclophosphamide as a component | |
| | | of adjuvant therapy in patients with | |
| | | evidence of axillary node tumor | |
| | | involvement following resection of | |
| | | primary breast cancer | |
| doxorubicin | Adriamycin, Rubex | | Pharmacia & Upjohn |
| | | | Company |
| doxorubicin | Adriamycin PFS | Antibiotic, antitumor agent. | Pharmacia & Upjohn |
| | Injectionintravenous | | Company |
| | injection | | |
| doxorubicin | Doxil | Conversion to regular approval for | Alza |
| liposomal | | treatment of patients with ovarian | |
| | | cancer whose disease has | |
| | | progressed or recurred after | |
| | | platinum-based chemotherapy | |
| doxorubicin | Doxil | Accel. Approv. (clinical benefit not | Sequus Pharmaceuticals, |
| liposomal | | established) Treatment of AIDS- | Inc. |
| | | related Kaposi's sarcoma in | |
| | | patients with disease that has | |
| | | progressed on prior combination | |
| | | chemotherapy or in patients who | |
| | | are intolerant to such therapy. | |
| doxorubicin | Doxil | Accel. Approv. (clinical benefit not | Sequus Pharmaceuticals, |
| liposomal | | established) Treatment of | Inc. |
| | | metastatic carcinoma of the ovary | |
| | | in patient with disease that is | |
| | | refractory to both paclitaxel and | |
| | | platinum based regimens | |
| DROMOSTANOLONE | DROMOSTANOLONE | | Eli Lilly |
| PROPIONATE | | | |
| DROMOSTANOLONE | MASTERONE | | SYNTEX |
| PROPIONATE | INJECTION | | |
| Elliott's B Solution | Elliott's B Solution | Diluent for the intrathecal | Orphan Medical, Inc |
| | | administration of methotrexate | |
| | | sodium and cytarabine for the | |
| | | prevention or treatment of | |
| | | meningeal leukemia or | |
| | | lymphocytic lymphoma. | |
| epirubicin | Ellence | A component of adjuvant therapy | Pharmacia & Upjohn |
| | | in patients with evidence of | Company |
| | | axillary node tumor involvement | |
| | | following resection of primary | |
| | | breast cancer. | |
| Epoetin alfa | epogen | EPOGENB is indicated for the | Amgen, Inc |
| | | treatment of anemic patients | |
| | | (hemoglobin >10 to _<13 g/dL) | |
| | | scheduled to undergo elective, | |
| | | noncardiac, nonvascular surgery to | |
| | | reduce the need for allogeneic | |
| | | blood transfusions. | |
| Epoetin alfa | epogen | EPOGENB is indicated for the | Amgen, Inc |
| | | treatment of anemia in patients | |
| | | with non-myeloid malignancies | |
| | | where anemia is due to the effect of | |
| | | concomitantly administered | |
| | | chemotherapy. EPOGEND is | |
| | | indicated to decrease the need for | |
| | | transfusions in patients who will be | |
| | | receiving concomitant | |
| | | chemotherapy for a minimum of 2 | |
| | | months. EPOGENB is not | |
| | | indicated for the treatment of | |
| | | anemia in cancer patients due to | |
| | | other factors such as iron or folate | |
| | | deficiencies, hemolysis or | |
| | | gastrointestinal bleeding, which | |
| | | should be managed appropriately. | |
| Epoetin alfa | epogen | EPOGEN is indicated for the | Amgen, Inch |
| | | treatment of anemia associated | |
| | | with CRF, including patients on | |
| | | dialysis (ESRD) and patients not | |
| | | on dialysis. | |
| erlotinib | Tarceva | For treatment of locally advanced | OSI |
| | | or metastatic Non Small-Cell Lung | |
| | | Cancer (NSCLC) after failure of at | |
| | | least one prior chemotherapy | |
| | | regimen | |
| erlotinib | Tarceva | For use in combination with | OSI |
| | | gemcitabine for the first-line | |
| | | treatment of patients with locally | |
| | | advanced, unresectable or | |
| | | metastatic pancreatic cancer | |
| estramustine | Emcyt | palliation of prostate cancer | Pharmacia & Upjohn |
| | | | Company |
| etoposide phosphate | Etopophos | Management of refractory | Bristol-Myers Squibb |
| | | testicular tumors, in combination | |
| | | with other approved | |
| | | chemotherapeutic agents. | |
| etoposide phosphate | Etopophos | Management of small cell lung | Bristol-Myers Squibb |
| | | cancer, first-line, in combination | |
| | | with other approved | |
| | | chemotherapeutic agents. | |
| etoposide phosphate | Etopophos | Management of refractory | Bristol-Myers Squibb |
| | | testicular tumors and small cell | |
| | | lung cancer. | |
| etoposide, VP-16 | Vepesid | Refractory testicular tumors-in | Bristol-Myers Squibb |
| | | combination therapy with other | |
| | | approved chemotherapeutic agents | |
| | | in patients with refractory testicular | |
| | | tumors who have already received | |
| | | appropriate surgical, | |
| | | chemotherapeutic and | |
| | | radiotherapeutic therapy. | |
| etoposide, VP-16 | VePesid | In combination with other | Bristol-Myers Squibb |
| | | approved chemotherapeutic agents | |
| | | as first line treatment in patients | |
| | | with small cell lung cancer. | |
| etoposide VP-16 | Vepesid | In combination with other | Bristol-Myers Squibb |
| | | approved chemotherapeutic agents | |
| | | as first line treatment in patients | |
| | | with small cell lung cancer. | |
| exemestane | Aromasin | For adjuvant treatment of | Pharmacia |
| | | postmenopausal women with | |
| | | estrogen-receptor positive early | |
| | | breast cancer who have received | |
| | | two to three years of tamoxifen and | |
| | | are switched to AROMASIN ® for | |
| | | completion of a total of five | |
| | | consecutive years of adjuvant | |
| | | hormonal therapy | |
| exemestane | Aromasin | Treatment of advance breast cancer | Pharmacia & Upjohn |
| | | in postmenopausal women whose | Company |
| | | disease has progressed following | |
| | | tamoxifen therapy. | |
| Filgrastim | Neupogen | NEUPOGEN is indicated to | Amgen, Inc |
| | | decrease the incidence of infection, | |
| | | as manifested by febrile | |
| | | neutropenia, in patients with | |
| | | nonmyeloid malignancies receiving | |
| | | myelosuppressive anticancer drugs | |
| | | associated with a significant | |
| | | incidence of severe neutropenia | |
| | | with fever. | |
| Filgrastim | Neupogen | NEUPOGEN is indicated for | Amgen, Inc |
| | | reducing the time to neutrophil | |
| | | recovery and the duration of fever, | |
| | | following induction or | |
| | | consolidation hemotherapy | |
| | | treatment of adults with AML. | |
| floxuridine | FUDR | | Roche |
| (intraarterial) | | | |
| fludarabine | Fludara | Palliative treatment of patients with | Berlex Laboratories Inc. |
| | | B-cell lymphocytic leukemia | |
| | | (CLL) who have not responded or | |
| | | have progressed during treatment | |
| | | with at least one standard | |
| | | alkylating agent containing | |
| | | regimen. | |
| fluorouracil, 5-FU | Adrucil | prolong survival in combination | ICN Puerto Rico |
| | | with leucovorin | |
| fulvestrant | Faslodex | the treatment of hormone receptor- | IPR |
| | | positive metastatic breast cancer in | |
| | | postmenopausal women with | |
| | | disease progression following | |
| | | antiestrogen therapy | |
| gefitinib | Iressa | Accel. Approv. (clinical benefit not | AstraZenca |
| | | established) as monotherapy for | |
| | | the treatment of patients with | |
| | | locally advanced or metastatic non- | |
| | | small cell lung cancer after failure | |
| | | of both platinum-based and | |
| | | docetaxel chemotherapies | |
| gemcitabine | Gemzar | Treatment of patients with locally | Eli Lilly |
| | | advanced (nonresectable stage II or | |
| | | III) or metastatic (stage IV) | |
| | | adenocarcinoma of the pancreas. | |
| | | Indicated for first-line treatment | |
| | | and for patients previously treated | |
| | | with a 5-fluorouracil-containing | |
| | | regimen. | |
| gemcitabine | Gemzar | For use in combination with | Eli Lilly |
| | | cisplatin for the first-line treatment | |
| | | of patients with inoperable, locally | |
| | | advanced (Stage IIIA or IIIB) or | |
| | | metastatic (Stage IV) non-small | |
| | | cell lung cancer. | |
| gemicitabine | Gemzar | For use in combination with | Lilly |
| | | paclitaxel for the first-line | |
| | | treatment of patients with | |
| | | metastatic breast cancer after | |
| | | failure of prior anthracycline- | |
| | | containing adjuvant chemotherapy, | |
| | | unless anthracyclines were | |
| | | clinically contraindicated | |
| gemtuzumab | Mylotarg | Accel. Approv. (clinical benefit not | Wyeth Ayerst |
| ozogamicin | | established) Treatment of CD33 | |
| | | positive acute myeloid leukemia in | |
| | | patients in first relapse who are 60 | |
| | | years of age or older and who are | |
| | | not considered candidates for | |
| | | cytotoxic chemotherapy. | |
| goserelin acetate | Zoladex | | AstraZeneca |
| | | | Pharmaceuticals |
| goserelin acetate | Zoladex Implant | Palliative treatment of advanced | AstraZeneca |
| | | breast cancer in pre- and | Pharmaceuticals |
| | | perimenopausal women. | |
| histrelin acetate | Histrelin implant | For the palliative treatment of | Valera |
| | | advanced prostate cancer | |
| hydroxyurea | Hydrea | | Bristol-Myers Squibb |
| hydroxyurea | Hydrea | Decrease need for transfusions in | Bristol-Myers Squibb |
| | | sickle cell anemia | |
| Ibritumomab | Zevalin | Accel. Approv. (clinical benefit not | IDEC Pharmaceuticals |
| Tiuxetan | | established) treatment of patients | Corp |
| | | with relapsed or refractory low- | |
| | | grade, follicular, or transformed B- | |
| | | cell non-Hodgkin's lymphoma, | |
| | | including patients with Rituximab | |
| | | refractory follicular non-Hodgkin's | |
| | | lymphoma. | |
| idarubicin | Idamycin | For use in combination with other | Adria Laboratories |
| | | approved antileukemic drugs for | |
| | | the treatment of acute myeloid | |
| | | leukemia (AML) in adults. | |
| idarubicin | Idamycin | In combination with other | Pharmacia & Upjohn |
| | | approved antileukemic drugs for | Company |
| | | the treatment of acute non- | |
| | | lymphocytic leukemia in adults. | |
| ifosfamide | IFEX | Third line chemotherapy of germ | Bristol-Myers Squibb |
| | | cell testicular cancer when used in | |
| | | combination with certain other | |
| | | approved antineoplastic agents. | |
| imatinib mesylate | Gleevec | Accel. Approv. (clinical benefit not | Novartis |
| | | established) Initial therapy of | |
| | | chronic myelogenous leukemia | |
| imatinib mesylate | Gleevec | Accel. Approv. (clinical benefit not | Novartis |
| | | established) metastatic or | |
| | | unresectable malignant | |
| | | gastrointestinal stromal tumors | |
| Imatinib mesylate | Gleevec | Accel. Approv. (clinical benefit not | Novartis |
| | | established) Treatment of patients | |
| | | with Kit (CD117) positive | |
| | | unresectable and/or metastatic | |
| | | malignant gastrointestinal stromal | |
| | | tumors (GIST). | |
| imatinib mesylate | Gleevec | Accel. Approv. (clinical benefit not | Novartis |
| | | established) Initial treatment of | |
| | | newly diagnosed Ph+ chronic | |
| | | myelogenous leukemia (CML). | |
| imatinib mesylate | Gleevec | Accel. Approv. (clinical benefit not | Novartis |
| | | established) for treatment of newly | |
| | | diagnosed adult patients with | |
| | | Philadelphia chromosome positive | |
| | | chronic myeloid leukemia (CML) | |
| | | in chronic phase. Follow-up is | |
| | | limited. Gleevec is also indicated | |
| | | for the treatment of patients with | |
| | | Philadelphia chromosome positive | |
| | | chronic myeloid leukemia (CML) | |
| | | in blast crisis, accelerated phase, or | |
| | | in chronic phase after failure of | |
| | | interferon-alpha therapy. There are | |
| | | no controlled trials demonstrating a | |
| | | clinical benefit, such as | |
| | | improvement in disease-related | |
| | | symptoms or increased survival in | |
| | | patients with CML blast crisis, | |
| | | accelerated phase or chronic phase | |
| | | after failure of alpha interferon. | |
| | | Gleevec is also indicated for the | |
| | | treatment of patients with Kit | |
| | | (CD117) positive unresectable | |
| | | and/or metastatic malignant | |
| | | gastrointestinal stromal tumors | |
| | | (GIST) | |
| imatinib mesylate | Gleevec | Accel. Approv. (clinical benefit not | Novartis |
| | | established) Treatment of pediatric | |
| | | patients with Ph+ chronic phase | |
| | | CML whose disease has recurred | |
| | | after stem cell transplant or who | |
| | | are resistant to interferon alpha | |
| | | therapy. | |
| imatinib mesylate | Gleevec | Conversion to regular approval for | Novartis |
| | | treatment of patients with | |
| | | Philadelphia chromosome positive | |
| | | chronic myeloid leukemia (CML) | |
| | | in blast crisis, accelerated phase, or | |
| | | in chronic phase after failure of | |
| | | interferon-alpha therapy | |
| interferon alfa 2a | Roferon A | Treatment of patients with hairy | Roche |
| | | cell leukemia | |
| interferon alfa 2a | Roferon A | Chronic phase, Philadelphia | Roche |
| | | chromosome positive chronic | |
| | | myelogenous leukemia (CML) | |
| | | patients who are minimally | |
| | | pretreated (within 1 year of | |
| | | diagnosis) | |
| Interferon alfa-2a | Roferon-A | | Hoffmann-La Roche Inc |
| Interferon alfa-2b | Intron A | Interferon alfa-2b, recombinant for | Schering Corp |
| | | Injection is indicated for the | |
| | | treatment of patients 18 years of | |
| | | age or older with hairy cell | |
| | | leukemia. | |
| Interferon alfa-2b | Intron A | Interferon alfa-2b, recombinant for | Schering Corp |
| | | Injection is indicated for | |
| | | intralesional treatment of selected | |
| | | patients 18 years of age or older | |
| | | with condylomata acuminata | |
| | | involving external surfaces of the | |
| | | genital and perianal areas. | |
| Interferon alfa-2b | Intron A | Interferon alfa-2b, recombinant for | Schering Corp |
| | | injection is indicated for the | |
| | | treatment of selected patients 18 | |
| | | years of age or older with AIDS- | |
| | | related Kaposi's Sarcoma. The | |
| | | likelihood of response to INTRON | |
| | | A therapy is greater in patients who | |
| | | are without systemic symptoms, | |
| | | who have limited | |
| | | lymphadenopathy and who have a | |
| | | relatively intact immune system as | |
| | | indicated by total CD4 Count. | |
| Interferon alfa-2b | Intron A | Interferon alfa-2b, recombinant for | Schering Corp |
| | | injection is indicated as adjuvant to | |
| | | surgical treatment in patients 18 | |
| | | years of age or older with | |
| | | malignant melanoma who are free | |
| | | of disease but at high risk for | |
| | | systemic recurrence within 56 days | |
| | | of surgery. | |
| Interferon alfa-2b | Intron A | Interferon alfa-2b, recombinant for | Schering Corp |
| | | Injection is indicated for the initial | |
| | | treatment of clinically aggressive | |
| | | follicular non-Hodgkin's | |
| | | Lymphoma in conjunction with | |
| | | anthracycline-containing | |
| | | combination chemotherapy in | |
| | | patients 18 years of age or older. | |
| Interferon alfa-2b | Intron A Intron A | | Schering Corp |
| irinotecan | Camptosar | Accel. Approv. (clinical benefit | Pharmacia & Upjohn |
| | | subsequently established) | Company |
| | | Treatment of patients with | |
| | | metastatic carcinoma of the colon | |
| | | or rectum whose disease has | |
| | | recurred or progressed following 5- | |
| | | FU-based therapy. | |
| irinotecan | Camptosar | Conversion to regular approval- | Pharmacia & Upjohn |
| | | treatment of metastatic carcinoma | Company |
| | | of the colon or rectum whose | |
| | | disease has recurred or progressed | |
| | | following 5-FU-based therapy. | |
| irinotecan | Camptosar | For first line treatment n | Pharmacia & Upjohn |
| | | combination with 5-FU/leucovorin | Company |
| | | of metastatic carcinoma of the | |
| | | colon or rectum. | |
| lenalidomide | Revlimid | for the treatment of patients with | Celgene |
| | | transfusion-dependent anemia due | |
| | | to Low- or Intermediate-1-risk | |
| | | myelodysplastic syndromes | |
| | | associated with a deletion 5q | |
| | | cytogenetic abnormality with or | |
| | | without additional cytogenetic | |
| | | abnormalities | |
| letrozole | Femara | Treatment of advanced breast | Novartis |
| | | cancer in postmenopausal women. | |
| letrozole | Femara | First-line treatment of | Novartis |
| | | postmenopausal women with | |
| | | hormone receptor positive or | |
| | | hormone receptor unknown locally | |
| | | advanced or metastatic breast | |
| | | cancer. | |
| letrozole | Femara | | Novartis |
| letrozole | Femara | Accel. Approv. (clinical benefit not | Novartis |
| | | established) for the extended | |
| | | adjuvant treatment of early breast | |
| | | cancer in postmenopausal women | |
| | | who have received five years of | |
| | | adjuvant tamoxifen therapy. | |
| leucovorin | Leucovorin | | Immunex Corporation |
| leucovorin | Leucovorin | | Immunex Corporation |
| leucovorin | Leucovorin | | Immunex Corporation |
| leucovorin | Leucovorin | In combination with fluorouracil to | Lederle Laboratories |
| | | prolong survival in the palliative | |
| | | treatment of patients with advanced | |
| | | colorectal cancer. | |
| Leuprolide Acetate | Eligard | palliative treatment of advanced | QLT USA |
| | | prostate cancer. | |
| levamisole | Ergamisol | Adjuvant treatment in combination | Janssen Research |
| | | with 5-fluorouracil after surgical | Foundation |
| | | resection in patients with Dukes' | |
| | | Stage C colon cancer. | |
| lomustine, CCNU | CeeBU | | Bristol-Myers Squibb |
| meclorethamine, | Mustargen | | Merck |
| nitrogen mustard | | | |
| megestrol acetate | Megace | | Bristol-Myers Squibb |
| melphalan, L-PAM | Alkeran | | GlaxoSmithKline |
| melphalan, L-PAM | Alkeran | Systemic administration for | GlaxoSmithKline |
| | | palliative treatment of patients with | |
| | | multiple myeloma for whom oral | |
| | | therapy is not appropriate. | |
| mercaptopurine, 6- | Purinethol | | GlaxoSmithKline |
| MP | | | |
| mesna | Mesnex tabs | Reducing the incidence of | Baxter |
| | | ifosfamide-induced hemorrhagic | |
| | | cystitis | |
| methotrexate | Methotrexate | | Lederle Laboratories |
| methotrexate | Methotrexate | | Lederle Laboratories |
| methotrexate | Methotrexate | | Lederle Laboratories |
| methotrexate | Methotrexate | | Lederle Laboratories |
| methotrexate | Methotrexate | osteosarcoma | Lederle Laboratories |
| methotrexate | Methotrexate | | Lederle Laboratories |
| methoxsalen | Uvadex | For the use of UVADEX with the | Therakos |
| | | UVAR Photopheresis System in | |
| | | the palliative treatment of the skin | |
| | | manifestations of cutaneous T-cell | |
| | | lymphoma (CTCL) that is | |
| | | unresponsive to other forms of | |
| | | treatment. | |
| mitomycin C | Mutamycin | | Bristol-Myers Squibb |
| mitomycin C | Mitozytrex | therapy of disseminated | Supergen |
| | | adenocarcinoma of the stomach or | |
| | | pancreas in proven combinations | |
| | | with other approved | |
| | | chemotherapeutic agents and as | |
| | | palliative treatment when other | |
| | | modalities have failed. | |
| mitotane | Lysodren | | Bristol-Myers Squibb |
| mitoxantrone | Novantrone | For use in combination with | Immunex Corporation |
| | | corticosteroids as initial | |
| | | chemotherapy for the treatment of | |
| | | patients with pain related to | |
| | | advanced hormone-refractory | |
| | | prostate cancer. | |
| mitoxantrone | Novantrone | For use with other approved drugs | Lederle Laboratories |
| | | in the initial therapy for acute | |
| | | nonlymphocytic leukemia (ANLL) | |
| | | in adults. | |
| nandrolone | Durabolin-50 | | Organon |
| phenpropionate | | | |
| nelarabine | Arranon | Accel. Approv. (clinical benefit not | GlaxoSmithKline |
| | | established) for the treatment of | |
| | | patients with T-cell acute | |
| | | lymphoblastic leukemia and T-cell | |
| | | lymphoblastic lymphoma whose | |
| | | disease has not responded to or has | |
| | | relapsed following treatment with | |
| | | at least two chemotherapy | |
| | | regimens | |
| Nofetumomab | Verluma | | Boehringer Ingelheim |
| | | | Pharma KG (formerly Dr. |
| | | | Karl Thomae GmbH) |
| Oprelvekin | Neumega | | Genetics Institute, Inc |
| oxaliplatin | Eloxatin | Accel. Approv. (clinical benefit not | Sanofi Synthelabo |
| | | established) in combination with | |
| | | infusional 5-FU/LV, is indicated | |
| | | for the treatment of patients with | |
| | | metastatic carcinoma of the colon | |
| | | or rectum whose disease has | |
| | | recurred or progressed during or | |
| | | within 6 months of completion of | |
| | | first line therapy with the | |
| | | combination of bolus 5-FU/LV and | |
| | | irinotecan. | |
| oxaliplatin | Eloxatin | Conversion to regular approval for | Sanofi Synthelabo |
| | | use in combination with infusional | |
| | | 5-Fluorouracil (5-FU) and | |
| | | Leucovorin (LV) for the treatment | |
| | | of patients previously untreated for | |
| | | advanced colorectal cancer | |
| oxaliplatin | Eloxatin | for use in combination with | Sanofi Synthelabo |
| | | infusional 5-FU/LV, for the | |
| | | adjuvant treatment of stage III | |
| | | colon cancer patients who have | |
| | | undergone complete resection of | |
| | | the primary tumor | |
| paclitaxel | Paxene | treatment of advanced AIDS- | Baker Norton |
| | | related Kaposi's sarcoma after | Pharmaceuticals, Inc |
| | | failure of first line or subsequent | |
| | | systemic chemotherapy | |
| paclitaxel | Taxol | Treatment of patients with | Bristol-Myers Squibb |
| | | metastatic carcinoma of the ovary | |
| | | after failure of first-line or | |
| | | subsequent chemotherapy. | |
| paclitaxel | Taxol | Treatment of breast cancer after | Bristol-Myers Squibb |
| | | failure of combination | |
| | | chemotherapy for metastatic | |
| | | disease or relapse within 6 months | |
| | | of adjuvant chemotherapy. Prior | |
| | | therapy should have included an | |
| | | anthracycline unless clinically | |
| | | contraindicated. | |
| paclitaxel | Taxol | New dosing regimen for patients | Bristol-Myers Squibb |
| | | who have failed initial or | |
| | | subsequent chemotherapy for | |
| | | metastatic carcinoma of the ovary | |
| paclitaxel | Taxol | second line therapy for AIDS | Bristol-Myers Squibb |
| | | related Kaposi's sarcoma. | |
| paclitaxel | Taxol | For first-line therapy for the | Bristol-Myers Squibb |
| | | treatment of advanced carcinoma | |
| | | of the ovary in combination with | |
| | | cisplatin. | |
| paclitaxel | Taxol | for use in combination with | Bristol-Myers Squibb |
| | | cisplatin, for the first-line treatment | |
| | | of non-small cell lung cancer in | |
| | | patients who are not candidates for | |
| | | potentially curative surgery and/or | |
| | | radiation therapy. | |
| paclitaxel | Taxol | For the adjuvant treatment of node- | Bristol-Myers Squibb |
| | | positive breast cancer administered | |
| | | sequentially to standard | |
| | | doxorubicin-containing | |
| | | combination therapy. | |
| paclitaxel | Taxol | First line ovarian cancer with 3 | Bristol-Myers Squibb |
| | | hour infusion. | |
| paclitaxel protein- | Abraxane | For the treatment of breast cancer | AM Bioscience |
| bound particles | | after failure of combination | |
| | | chemotherapy for metastatic | |
| | | disease or relapse within 6 months | |
| | | of adjuvant chemotherapy. Prior | |
| | | therapy should have included an | |
| | | anthracyline unless clinically | |
| | | contraindicated | |
| pamidronate | Aredia | Treatment of osteolytic bone | Novartis |
| | | metastases of breast cancer in | |
| | | conjunction with standard | |
| | | antineoplastic therapy. | |
| pegademase | Adagen | Enzyme replacement therapy for | Enzon |
| | (Pegademase | patients with severe combined | |
| | Bovine) | immunodeficiency asa result of | |
| | | adenosine deaminase deficiency. | |
| pegaspargase | Oncaspar | Acute lymphocytic leukemia in L- | Enzon, Inc |
| | | asparaginase hypersensitive | |
| | | patients | |
| Pegfilgrastim | Neulasta | Neulasta is indicated to decrease | Amgen, Inc |
| | | the incidence of infection, as | |
| | | manifested by febrile neutropenia, | |
| | | in patients with non-myeloid | |
| | | malignancies receiving | |
| | | myelosuppressive anti-cancer | |
| | | drugs associated with a clinically | |
| | | significant incidence of febrile | |
| | | neutropenia. | |
| pemetrexed disodium | Alimta | For use in the treatment of patients | Lilly |
| | | with malignant pleural | |
| | | mesothelioma whose disease is | |
| | | either unresectable or who are | |
| | | otherwise not candidates for | |
| | | curative surgery | |
| pemetrexed disodium | Alimta | Accel. Approv. (clinical benefit not | Lilly |
| | | established) as a single agent for | |
| | | the treatment of patients with | |
| | | locally advanced or metastatic non- | |
| | | small lung cancer after prior | |
| | | chemotherapy | |
| pentostatin | Nipent | Single agent treatment for adult | Parke-Davis |
| | | patients with alpha interferon | Pharmaceutical Co. |
| | | refractory hairy cell leukemia. | |
| pentostatin | Nipent | Single-agent treatment for | Parke-Davis |
| | | untreated hairy cell leukemia | Pharmaceutical Co. |
| | | patients with active disease as | |
| | | defined by clinically significant | |
| | | anemia, neutropenia, | |
| | | thrombocytopenia, or disease- | |
| | | related symptoms. (Supplement for | |
| | | front-line therapy.) | |
| pipobroman | Vercyte | | Abbott Labs |
| plicamycin, | Mithracin | | Pfizer Labs |
| mithramycin | | | |
| porfimer sodium | Photofrin | For the ablation of high-grade | Axcan Scandipharm |
| | | dysplasia in Barrett's esophagus | |
| | | patients who do not undergo | |
| | | esophagectomy | |
| porfimer sodium | Photofrin | For use in photodynamic therapy | QLT Phototherapeutics Inc. |
| | | (PDT) for palliation of patients | |
| | | with completely obstructing | |
| | | esophageal cancer, or patients with | |
| | | partially obstructing esophageal | |
| | | cancer who cannot be satisfactorily | |
| | | treated with ND-YAG laser | |
| | | therapy. | |
| porfimer sodium | Photofrin | For use in photodynamic therapy | QLT Phototherapeutics Inc. |
| | | for treatment of microinvasive | |
| | | endobronchial nonsmall cell lung | |
| | | cancer in patients for whom | |
| | | surgery and radiotherapy are not | |
| | | indicated. | |
| porfimer sodium | Photofrin | For use in photodynamic therapy | QLT Phototherapeutics Inc. |
| | | (PDT) for reduction of obstruction | |
| | | and palliation of symptoms in | |
| | | patients with completely or | |
| | | partially obstructing endobroncial | |
| | | nonsmall cell lung cancer | |
| | | (NSCLC). | |
| procarbazine | Matulane | | Sigma Tau Pharms |
| quinacrine | Atabrine | | Abbott Labs |
| Rituximab | Rituxan | for use in the first-line treatment of | Genentech, Inc |
| | | patients with diffuse large B-cell, | |
| | | CD20-positive, non-Hodgkin's | |
| | | lymphoma in combination with | |
| | | CHOP or other anthracycline-based | |
| | | chemotherapy regimens. | |
| Rituximab | Rituxan | Treatment of patients with relapsed | Genentech, Inc |
| | | or refractory low-grade or | |
| | | follicular B-cell non-Hodgkin's | |
| | | lymphoma | |
| Sargramostim | Prokine | | Immunex Corp |
| sorafenib | Nexavar | For the treatment of patients with | Bayer |
| | | advanced renal cell carcinoma | |
| streptozocin | Zanosar | Antineolastic agent. | Pharmacia & Upjohn |
| | | | Company |
| sunitinib maleate | Sutent | treatment of gastrointestinal | Pfizer |
| | | stromal tumor after disease | |
| | | progression on or intolerance to | |
| | | imatinib mesylate | |
| sunitinib maleate | Sutent | Accel. Approv. (clinical benefit not | Pfizer |
| | | established) for the treatment of | |
| | | advanced renal cell carcinoma. | |
| | | Approval for advanced renal cell | |
| | | carcinoma is based on partial | |
| | | response rates and duration of | |
| | | responses. There are no | |
| | | randomized trials of SUTENT | |
| | | demonstrating clinical benefit such | |
| | | as increased survival or | |
| | | improvement in disease-related | |
| | | symptoms in renal cell carcinoma. | |
| talc | Sclerosol | For the prevention of the | Bryan |
| | | recurrence of malignant pleural | |
| | | effusion in symptomatic patients. | |
| tamoxifen | Nolvadex | | AstraZeneca |
| | | | Pharmaceuticals |
| tamoxifen | Nolvadex | As a single agent to delay breast | AstraZeneca |
| | | cancer recurrence following total | Pharmaceuticals |
| | | mastectomy and axillary dissection | |
| | | in postmenopausal women with | |
| | | breast cancer (T1-3, N1, M0) | |
| tamoxifen | Nolvadex | For use in premenopausal women | AstraZeneca |
| | | with metastatic breast cancer as an | Pharmaceuticals |
| | | alternative to oophorectomy or | |
| | | ovarian irradiation | |
| tamoxifen | Nolvadex | For use in women with axillary | AstraZeneca |
| | | node-negative breast cancer | Pharmaceuticals |
| | | adjuvant therapy. | |
| tamoxifen | Nolvadex | Metastatic breast cancer in men. | AstraZeneca |
| | | | Pharmaceuticals |
| tamoxifen | Nolvadex | Equal bioavailability of a 20 mg | AstraZeneca |
| | | Nolvadex tablet taken once a day | Pharmaceuticals |
| | | to a 10 mg Nolvadex tablet taken | |
| | | twice a day. | |
| tamoxifen | Nolvadex | to reduce the incidence of breast | AstraZeneca |
| | | cancer in women at high risk for | Pharmaceuticals |
| | | breast cancer | |
| tamoxifen | Nolvadex | In women with DCIS, following | AstraZeneca |
| | | breast surgery and radiation, | Pharmaceuticals |
| | | Nolvadex is indicated to reduce the | |
| | | risk of invasive breast cancer. | |
| temozolomide | Temodar | Accel. Approv. (clinical benefit not | Schering |
| | | established) Treatment of adult | |
| | | patients with refractory anaplastic | |
| | | astrocytoma, i.e., patients at first | |
| | | relapse with disease progression on | |
| | | a nitrosourea and procarbazine | |
| | | containing regimen | |
| temozolomide | Temodar | Conversion to regular approval for | Schering |
| | | the treatment of patients with | |
| | | newly diagnosed high grade | |
| | | gliomas concomitantly with | |
| | | radiotherapy and then as adjuvant | |
| | | treatment | |
| teniposide, VM-26 | Vumon | In combination with other | Bristol-Myers Squibb |
| | | approved anticancer agents for | |
| | | induction therapy in patients with | |
| | | refractory childhood acute | |
| | | lymphoblastic leukemia (all). | |
| testolactone | Teslac | | Bristol-Myers Squibb |
| testolactone | Teslac | | Bristol-Myers Squibb |
| thioguanine, 6-TG | Thioguanine | | GlaxoSmithKline |
| thiotepa | Thioplex | | Immunex Corporation |
| thiotepa | Thioplex | | Immunex Corporation |
| thiotepa | Thioplex | | Lederle Laboratories |
| topotecan | Hycamtin | Treatment of patients with | GlaxoSmithKline |
| | | metastatic carcinoma of the ovary | |
| | | after failure of initial or subsequent | |
| | | chemotherapy. | |
| topotecan | Hycamtin | Treatment of small cell lung cancer | GlaxoSmithKline |
| | | sensitive disease after failure of | |
| | | first-line chemotherapy. In clinical | |
| | | studies submitted to support | |
| | | approval, sensitive disease was | |
| | | defined as disease responding to | |
| | | chemotherapy but subsequently | |
| | | progressing at least 60 days (in the | |
| | | phase 3 study) or at least 90 days | |
| | | (in the phase 2 studies) after | |
| | | chemotherapy | |
| toremifene | Fareston | Treatment of advanced breast | Orion Corp. |
| | | cancer in postmenopausal women. | |
| Tositumomab | Bexxar | Accel. Approv. (clinical benefit not | Corixa Corporation |
| | | established) Treatment of patients | |
| | | with CD20 positive, follicular, | |
| | | non-Hodgkin's lymphoma, with | |
| | | and without transformation, whose | |
| | | disease is refractory to Rituximab | |
| | | and has relapsed following | |
| | | chemotherapy | |
| Tositumomab/I-131 | Bexxar | Expand the indication to include | GlaxoSmithKline |
| tositumomab | | patients with relapsed or refractory | |
| | | low grade follicular transformed | |
| | | CD20-positive non-Hodgkin's | |
| | | lymphoma who have not received | |
| | | rituximab | |
| Trastuzumab | Herceptin | HERCEPTIN as a single agent is | Genentech, Inc |
| | | indicated for the treatment of | |
| | | patients with metastatic breast | |
| | | cancer whose tumors overexpress | |
| | | the HER2 protein and who have | |
| | | received one or more | |
| | | chemotherapy regimens for their | |
| | | metastatic disease. | |
| Trastuzumab | Herceptin | Herceptin in combination with | Genentech, Inc |
| | | paclitaxel is indicated for treatment | |
| | | of patients with metastatic breast | |
| | | cancer whose tumors overexpress | |
| | | the HER-2 protein and had not | |
| | | received chemotherapy for their | |
| | | metastatic disease | |
| Trastuzumab | Herceptin | | Genentech, Inc |
| Trastuzumab | Herceptin | | Genentech, Inc |
| tretinoin, ATRA | Vesanoid | Induction of remission in patients | Roche |
| | | with acute promyelocytic leukemia | |
| | | (APL) who are refractory to or | |
| | | unable to tolerate anthracycline | |
| | | based cytotoxic chemotherapeutic | |
| | | regimens. | |
| Uracil Mustard | Uracil Mustard | | Roberts Labs |
| | Capsules | | |
| valrubicin | Valstar | For intravesical therapy of BCG- | Anthra --> Medeva |
| | | refractory carcinoma in situ (CIS) | |
| | | of the urinary bladder in patients | |
| | | for whom immediate cystectomy | |
| | | would be associated with | |
| | | unacceptable morbidity or | |
| | | mortality. | |
| vinblastine | Velban | | Eli Lilly |
| vincristine | Oncovin | | Eli Lilly |
| vincristine | Oncovin | | Eli Lilly |
| vincristine | Oncovin | | Eli Lilly |
| vincristine | Oncovin | | Eli Lilly |
| vincristine | Oncovin | | Eli Lilly |
| vincristine | Oncovin | | Eli Lilly |
| vincristine | Oncovin | | Eli Lilly |
| vinorelbine | Navelbine | Single agent or in combination | GlaxoSmithKline |
| | | with cisplatin for the first-line | |
| | | treatment of ambulatory patients | |
| | | with unresectable, advanced non- | |
| | | small cell lung cancer (NSCLC). | |
| vinorelbine | Navelbine | Navelbine is indicated as a single | GlaxoSmithKline |
| | | agent or in combination with | |
| | | cisplatin for the first-line treatment | |
| | | of ambulatory patients with | |
| | | unreseactable, advanced non-small | |
| | | cell lung cancer (NSCLC). In | |
| | | patients with Stage IV NSCLC, | |
| | | Navelbine is indicated as a single | |
| | | agent or in combination with | |
| | | cisplatin. In Stage III NSCLC, | |
| | | Navelbine is indicated in | |
| | | combination with cisplatin. | |
| zoledronate | Zometa | the treatment of patients with | Novartis |
| | | multiple myeloma and patients | |
| | | with documented bone metastases | |
| | | from solid tumors, in conjunction | |
| | | with standard antineoplastic | |
| | | therapy. Prostate cancer should | |
| | | have progressed after treatment | |
| | | with at least one hormonal therapy | |
| zoledronic acid | Zometa | Treatment of hypercalcemia of | Novartis |
| | | malignancy | |

The chemotherapeutic agent of the present invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the chemotherapeutic agent accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients together with the alpha radionuclides are contained in an amount effective to achieve the intended purpose. More specifically, a therapeutically effective amount means an amount of active ingredients (chemotherapeutic agent), which together with the alpha emitting radionuclides of the present invention are effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose of the chemotherapeutic agent and the alpha radionucleide can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals, such as those described herein below. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide so that the active ingredient are sufficient to induce or suppress the biological effect (minimal effective concentration, MEC) and to cause a synergistic effect. The MEC will vary for each preparation, but can be estimated from in vitro data. Dosages necessary to achieve the MEC will depend on individual characteristics and route of administration. Detection assays can be used to determine plasma concentrations.

Below is a list of animal models and cell lines that may be used to assay the combined effect of alpha radiation and a chemotherapeutic agent:

Tumor formation in transgenic mice overexpressing an oncogene—A transgenic mouse model for cancer (e.g., breast cancer) such as the erb model (Shah N., et al., 1999, Cancer Lett. 146: 15-2; Weistein E J., et al., 2000, Mol. Med. 6: 4-16) or MTV/myc model (Stewart T A et al., 1984, Cell, 38: 627-637), the c-myc model (Leder A., et al., 1986, Cell, 45:485-495), v-Ha-ras or c-neu model (Elson A and Leder P, 1995, J. Biol. Chem. 270: 26116-22) can be used to test the ability of alpha emitting radionuclides and a chemotherapeutic agent to suppress tumor growth in vivo.

Tumor formation in mice administered with cancerous cell lines—For the formation of solid tumors, athymic mice can be injected with non-mouse cancerous cells (e.g. human cancerous cells), and normal mice can be injected with mouse derived cancer cells, such as those derived from breast cancer, colon cancer, ovarian cancer, prostate cancer or thyroid cancer, and following the formation of cancerous tumors, the mice can be subjected to intra-tumor administration of alpha emitting radionuclides and to intra-tumor/or systemic administration of the chemotherapeutic agent.

The following cell lines (provided with their ATCC Accession numbers) can be used for each type of cancer model:

For breast cancer:

Human breast cancer cell lines—MDA-MB-453 (ATCC No. HTB-131), MDA-MB-231 (ATCC No. HTB-26), BT474 (ATCC No. HTB-20), MCF-7 (ATCC No. HTB-22), MDA-MB-468, (for additional cell lines see http://wwwdotpathdotcamdotacdotuk/˜pawefish/index.html);

For ovarian cancer:

Human ovarian cancer cell lines—SKOV3 (ATCC No. HTB-77), OVCAR-3 HTB-161), OVCAR-4, OVCAR-5, OVCAR-8 and IGROV1;

For prostate cancer:

Human prostate cancer cell lines—DU-145 (ATCC No. HTB-81), PC-3 (ATCC No. CRL-1435);

For thyroid cancer:

Human derived thyroid cancer cell lines—FTC-133, K1, K2, NPA87, K5, WRO82-1, ARO89-1, DRO81-1;

For lung cancer:

Mouse lung carcinoma LL/2 (LLC1) cells (Lewis lung carcinoma)—These cells are derived from a mouse bearing a tumor resulting from an implantation of primary Lewis lung carcinoma. The cells are tumorigenic in C57BL mice, express H-2b antigen and are widely used as a model for metastasis and for studying the mechanisms of cancer chemotherapeutic agents (Bertram J S, et al., 1980, Cancer Lett. 11: 63-73; Sharma S, et al. 1999, J. Immunol. 163: 5020-5028).

Culturing conditions of cancerous cells—The cancerous cells can be cultured in a tissue culture medium such as the DMEM with 4 mM L-glutamine adjusted to contain 1.5 g/L sodium bicarbonate and 4.5 g/L glucose, supplemented with 10% fetal calf serum (FCS), according to known procedures (e.g., as described in the ATCC protocols).

Tumor formation in animal models by administration of cancerous cells—Athymic nu/nu mice (e.g., female mice) can be purchased from the Jackson Laboratory (Bar Harbor, Me.). Tumors can be formed by subcutaneous (s.c.) injection of cancerous cells (e.g., 2×10⁶ cells in 100 μl of PBS per mouse). Tumors are then allowed to grow in vivo for several days (e.g., 6-14 days) until they reach a detectable size of about 0.5 cm in diameter. It will be appreciated that injection of cancerous cells to an animal model can be at the organ from which the cell line is derived (e.g., mammary tissue for breast cancer, ovary for ovarian cancer) or can be injected at an irrelevant tissue such as the rear leg of the mouse.

Modes of administration of chemotherapeutic agents to tumor—To test the effect of the chemotherapeutic agent and alpha emitting radionuclides on inhibition of tumor growth, the chemotherapeutic agent is administered to the animal model bearing the tumor either locally at the site of tumor or systemically, by intravenous injection of infusion, via, e.g., the tail vein. The time of administration of the chemotherapeutic agent may vary from immediately following injection of the cancerous cell line (e.g., by systemic administration) or at predetermined time periods following the appearance of the solid tumor (e.g., to the site of tumor formation, every 3 days for 3-10 times as described in Ugen K E et al., Cancer Gene Ther. Jun. 9, 2006; [Epub ahead of print]).

Evaluation of solid tumor inhibition—Tumor sizes are measured two to three times a week. Tumor volumes are calculated using the length and width of the tumor (in millimeters). The effect of the combined treatment can be evaluated by comparing the tumor volume using statistical analyses such as Student's t test. In addition, histological analyses can be performed using markers typical for each type of cancer.

Altogether, once the tumors are formed, the chemotherapeutic agent and the alpha emitting radionuclides are administered to the individual in need thereof, e.g., the animal model bearing the tumor, either locally or systemically, and the effect of the agent on tumor growth is detected using methods known in the art.

Depending on the severity and responsiveness of the condition to be treated, dosing can be of a single or a plurality of administrations, with course of treatment lasting until cure is effected or diminution of the disease state is achieved.

The amount of radiation and composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings at any age which suffer from the pathology. Preferably, this term encompasses individuals who are at risk to develop the pathology.

It is expected that during the life of a patent maturing from this application many relevant chemotherapeutic agents will be developed and the scope of the term chemotherapeutic agent is intended to include all such new technologies a priori.

As used herein the term “about” refers to ±10%

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

General Materials and Methods

Tumors: SQ2 cell line is a murine anaplastic cell line, which was generated from a SCC tumor that has developed spontaneously in a male BALB/c mouse. Panc02 is a murine pancreatic carcinoma cell line. CT26 cells is a N-nitroso-N-methylurethane-(NNMU) induced, undifferentiated colon carcinoma cell line which was purchased from the ATCC (CRL-2638). All cells were grown in Dulbecco's Modified Eagle Medium (DMEM) supplemented with 10% Fetal Calf Serum (Biological Industries, Beit Haemek, Israel), L-glutamine (2 mM), Penicillin (100 U/ml) and Streptomycin (100 μg/ml).

Radioactive microplates: A set-up was developed in which a regular 96-well microplate (Corning, Corning, USA) underwent ²²⁴Ra implantation using small ²²⁸Th panels corresponding in size to the bottom of the wells. The implantation was executed inside a vacuum chamber, using an eight headed stamp fitting a single column of the microplate. By controlling the time of the radioactive exposure, it was possible to determine the intensity of ²²⁴Ra atoms implanted in each column of wells.

Cell proliferation assay: The antiproliferative effects of alpha particles and cisplatin, alone and in combination, were determined using a 3-bis (2-methoxy-4-nitro-5 sulfenyl)-(2H)-tetrazolium-5-carboxanilide (XTT) assay (Cell Proliferation Kit, Biological industries, Beit-haemek, Israel). Cells (10⁴ per well) were seeded in 96-well microplates implanted with increasing intensities of ²²⁴Ra atoms (radioactive microplates). Cells were allowed to grow for the required period of time following which, the activated XTT mixture was added to a final concentration of 0.33 mg/ml according to the manufacturer's instructions. After two hours of incubation, absorbance was analyzed using an automated spectrophotometer (VersaMax, Molecular Devices, USA) at 475 nm wavelength.

Kapton wells set-up: Cells seeded on a thin (7.5 μm) Kapton (polyimide) foil were exposed to alpha particles traversing the foil from below. The set-up comprised of two stainless steel rings identical in diameter (35 mm) with a centered hole of 9 mm. One of the rings was 3 mm high, while the second was 12 mm high. The kapton foil (Dupont, Luxembourg) was placed between the two rings (the 12 mm ring on the top) covering the hole, and the rings were then screwed tightly and a rubber O-ring insured impermeability. After UV light sterilization of the wells (at least 1 hour), cells were seeded on the foil at a density of 5·10⁴ cells/well and exposed to the alpha particle flux 24 hours later. Exposure was performed by positioning the cells seeded on the foil 10 mm above a silicon wafer coated with a thin layer of ²²⁸Th in secular equilibrium with its daughters (collimated by a 10 mm circular hole) in air. The average alpha particle flux across the kapton foil was measured by an EG&G solid-state alpha particle detector. Exposure times were 0, 1, and 3 minutes, with an average flux of 1.1·10⁴ alpha particles/mm²·min across the exposed area. The calculated average dose rate, based on a Monte-Carlo calculation (not shown) performed using the SRIM-2003 code, was 0.8 Gy/min.

Annexin V/propidium iodide (PI) apoptosis assay: In order to detect the fraction of apoptotic cells, an Annexin-V/PI assay (MBL, Naka-ku Nagoya, Japan) was used. The SQ2 cells were seeded in kapton wells as described above, and treated either with cisplatin or alpha particles flux or a combination of the two modalities. Four hours following treatment, cells were collected using trypsin and washed once with PBS followed by another wash with binding buffer. The cells were incubated with 10 μL Annexin-V-fluorescein isothiocyanate (FITC) and 5 μL PI in the dark for 15 minutes and analyzed in a flow cytometer (Facsort, Becton Dickinson, USA).

Animals: Male BALB/c and female C57BL/6 mice (8-12 weeks old) were used. All surgical and invasive procedures were performed under anesthesia by Intra-peritoneal inoculation of imalgen (100 mg/kg, Fort Dodge, USA) and xylazine hydrochloride (10 mg/kg, VMD, Belgium) solution in 0.25 ml of PBS.

Tumor cell inoculation: Animals were inoculated intra-cutaneously with 5·10⁵ SQ2 cells in 0.2 ml HBSS or 10⁵ (CT26 and Panc-02) in 0.1 ml of HBSS (Biological industries, Beit haemek, Israel) into the low lateral side of the back. Local tumor growth was determined by measuring three mutually orthogonal tumor diameters with a digital caliper (Mitutoyo, Japan). The volume of tumor was calculated using the formula: V=(π/6)·D₁D₂D₃, where D₁, D₂, D₃ stand for the measured diameters

²²⁴Ra wire (DART wire) preparation: ²²⁴Ra wires were prepared as described in US Patent Application Publication No. 2007-0041900 to Kelson et al, incorporated herein by reference. Such a wire is a radiotherapy device, comprising a probe adapted for being at least partially introduced into a body of a subject, and an alpha emitting radionuclide. The radionuclide is on or beneath a surface of the probe, such that decay chain nuclei and alpha particles of the radionuclide are emitted outside the surface.

To prepare the wires, positive ²²⁴Ra ions emitted by recoil from a surface layer containing ²²⁸Th, were electrostatically collected near the tip of a thin conducting wire (0.3 mm in diameter) stainless steel needle. The wires were then heat-treated to induce radium diffusion away from the surface, to a typical depth of 10-20 nanometers. The ²²⁴Ra-impregnated wires were then characterized by an alpha particle detector to account for their ²²⁴Ra activity and release rate of ²²⁰Rn. The wires used in the in-vivo experiments had ²²⁴Ra activities in the range of 10-30 kBq, with ²²⁰Rn desorption probabilities of 22-36%.

Wire insertion: Wires, either loaded with ²²⁴Ra or inert, cut to a length of 5-6 mm, were placed near the tip of a 23G needle attached to a 2.5 ml syringe (Picindolor, Rome, Italy) and inserted into the tumor by a plunger placed internally along the syringe axis.

Histology: Histological analysis was performed on BALB/c mice lungs, both treated and untreated. Immediately following their removal, lungs were fixed by a 4% formaldehyde solution (Sigma, Rehovot, Israel) for at least 24 hrs. The preserved specimens were embedded in paraffin, and sections (5-10 μm) were stained with hematoxylin-eosin (H&E) (Surgipath, Richmond, USA) and analyzed for metastases detection. Metastatic burden quantification was performed by summing the gray values of all the pixels in the region of interest (ROI) divided by the number of pixels using image J free software [http://rsbdotinfodotnihdotgov/ij/].

Statistical analysis: The statistical significance (p-value) of the differences between tumor volumes in the various groups was assessed by applying Student's two-sided t-test and repeated measures ANOVA. Survival analysis (Mantel-Cox test) was carried out using Statsoft Statistica 7.0.

### Example 1

### Combination Between Alpha Particles and Cisplatin Enhanced Squamous Cell Carcinoma Cell Death and Arrested Proliferation in Culture

The following experiment was performed in order to determine whether cells treated with a combined strategy is more effective than a single treatment.

SQ2 cells were plated in 96 well plates implanted with ²²⁴Ra atoms (0, 0.02, 0.063, 0.2, 0.63 and 2 Bq/mm², radioactive microplates). For each radioactive dose, 3 concentrations of cisplatin were added to the microplate (0.3, 3, 30 μM). Cell numbers were assessed by the XTT assay 24, 48, and 72 hrs of incubation and expressed as percent of non-treated control cells.

FIGS. 1A-B show the observed inhibition effect of alpha particles and cisplatin on SQ2 cell proliferation at 48 hours (FIG. 1A) and 72 hours (FIG. 1B).

As can be seen in FIGS. 1A-B, substantial proliferation arrest caused by alpha irradiation alone was detected after 48 hrs, and the effect intensified after 72 hrs.

A dose dependent inhibition for cell growth effect was observed and ranged from 18% in wells exposed to 0.63 Bq/mm² up to 52% inhibition in 2 Bq/mm² wells, incubated for 72 hours.

An anti-proliferative effect was observed for cells incubated with various amounts of cisplatin alone.

A similar but stronger anti-proliferative effect was observed for cells incubated with 0.3 μM cisplatin and radioactivity. A higher proliferation inhibition, as shown in FIGS. 1A-B, was evident after 48 and 72 hours. Cells exposed to 0.2 Bq/mm² for 72 hours showed 18% inhibition and 0.3 μM of cisplatin caused 21%. However, the combined treatment gave rise to 34% proliferation arrest. At higher levels of the drug (3 and 30 μM) a strong antiproliferative effect (>60%) was induced by the drug alone, and obscured any additive effects with alpha radiation.

### Example 2

### Combination Between Alpha Particles and Cisplatin Induced Apoptosis in Squamous Cell Carcinoma Cells

Apoptotic cell death was monitored by the Annexin V dye-binding assay. Cells were co-stained with propidium iodide, which permeates into dead cells, to distinguish apoptotic cells from necrotic cells. Cells seeded in the kapton wells were exposed to two doses of alpha irradiation (0.8 Gy and 2.4 Gy) with or without cisplatin (30 μM), and compared to treatment by cisplatin only or non-treated cells (see Wang, X. B. et al. J Biochem 2004 135:555-565). FIG. 2G shows the percentage of apoptotic cells in all treated cultures. Less then 16% of untreated cells were positively stained by annexin V, and only a moderate increase was detected for cells irradiated by 0.8 Gy (19%). When cells were exposed to 2.4 Gy or to the chemotherapeutic agent alone, the level of apoptosis increase (22-23%). Furthermore, when chemotherapy and alpha-radiation were applied together, the apoptotic fraction increased for both radioactivity dose levels; 27% for CP+0.8 Gy and 41% for CP+2.4 Gy.

### Example 3

### Single DART Wire Insertion Combined with Two Cisplatin Treatments Moderately Retarded Squamous Cell Carcinoma Tumor Growth

This experiment was performed in order to study the effect of the combination of ²²⁴Ra wire inserted into tumors and cisplatin given intravenously in BALB/c mice bearing SQ2 tumors.

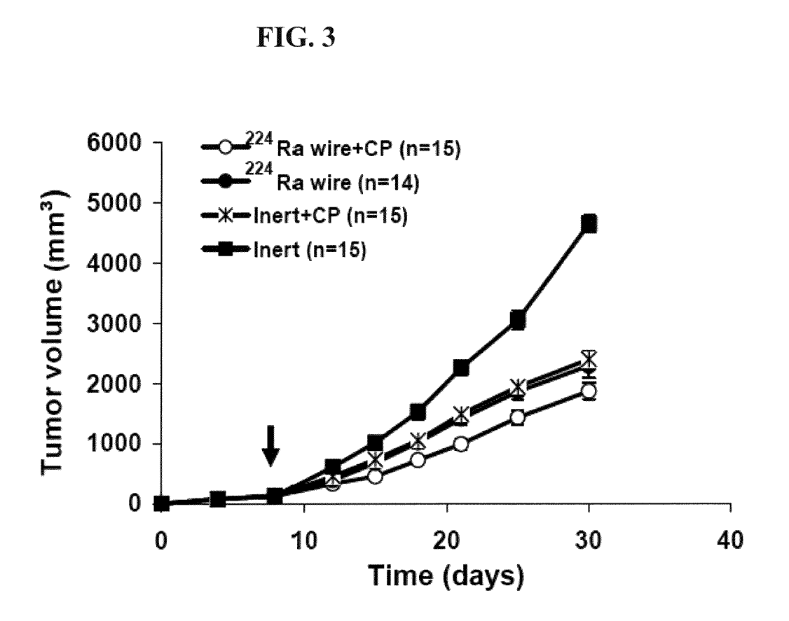

The DART wire treatment was executed as tumors reached the average size of 6-7 mm in diameter. The chemotherapeutic agent was injected in two separate doses of 5 mg/kg per animal—the first dose was administrated one day prior to DART treatment and the second was given 5 days later. Inert (non-radioactive) wires identical in shape to the radioactive ones were used as controls. The outcome of this line of experiments, as illustrated in FIG. 3, suggests that both α—radiation and chemotherapy (²²⁴Ra wire and CP groups) contribute to tumor growth retardation as stand-alone treatments. Average tumor volumes 30 days after tumors transplantation were very similar for both treatment groups (48-51% of the inert control group). When evaluating the joint effect yielded by the combined treatment group (²²⁴Ra wire+CP) it appeared that the average tumor volumes were smaller than in each treatment alone (40% of the inert control group on day 30), but the differences were not statistically significant (p values between the combination group and the CP or ²²⁴Ra wire groups were 0.054 and 0.105 respectively).

### Example 4

### Insertion of Two DART Wires Combined with Two Cisplatin Doses Significantly Retarded Squamous Cell Carcinoma Tumor Growth and Prolonged Survival

The following experiment was performed in order to examine the effect of two ²²⁴Ra wires inserted horizontally to the base of each tumor in combination with 2 doses of chemotherapy. The cisplatin was administered using the same regime as that described in Example 3.

The double ²²⁴Ra wire insertion had a prominent effect on tumor development as shown in FIG. 4A. A pronounced difference between tumor volumes of the irradiated group (²²⁴Ra wires) and the animals treated with Cisplatin (CP) can be seen 10 days following DART treatment. This difference became more evident with time, and 32 days after tumor cell inoculation the average tumor volume of the CP group was 2.14 fold greater than the DART treated group. Moreover, when analyzing the results of the group treated with the combination of Cisplatin and ²²⁴Ra wires, it appears that a major growth inhibition was achieved when both modalities were administrated concomitantly. Over 50% of the animals in the combination group showed tumor retardation at some point of the monitoring, with complete tumor eradication in one case. Twenty-four days following DART treatment, the average tumor volumes of the combined treatment group was 14 fold smaller compared to the inert control group (300 mm³ and 4286 mm³ respectively), and 3 fold smaller compared to the best effect achieved by the radioactive wires alone (924 mm³). A survival follow-up was done on all 4 tested groups in order to examine the differences in effects on life expectancy between treatments. The findings presented in FIG. 4B indicate that all three treatments prolonged life span significantly. A more thorough examination revealed that even though mice that got treated with cisplatin alone survived longer than the control group (Mean survival of 51.38 days and 43.92 days respectively, p=0.0093 ), the treatment group which received ²²⁴Ra wires survived even longer (66.5 days, p=0.00001). Moreover, the integration of both Cisplatin and intratumoral radioactive wires yielded a pronounced and significant larger effect on life expectancy. While both stand-alone treatments prolonged average survival by 17% and 51% (chemotherapy and radiotherapy respectively), the combination between the two almost doubled animals average life span (87.27 days-98% compared to inert group).

Thus, the survival prolongation of the combined therapy was much higher than the sum of prolongation achieved with each therapy alone.

### Example 5

### Insertion of Two DART Wires Combined with Two Cisplatin Doses Reduced Metastatic Load in the Lungs of Squamous Cell Carcinoma Bearing Mice

Histological assessment of lung sections was conducted in order to investigate the effect of the destruction of the primary tumor by DART wires on the development of metastases with or without the addition of cisplatin. Each 4 groups (Inert, ²²⁴Ra wires, CP, CP+²²⁴Ra wires) contained 3 animals. Animals were sacrificed at day 26 (lung metastases has been observed in this model at this time in previous studies) and lungs were harvested and processed for histological analysis and compared to normal lung tissues taken from healthy BALB/c mice. FIG. 5C describes the inhibition of lung metastatic load in mice treated with both intratumoral alpha irradiation and chemotherapy when compared to lungs of mice treated with inert wires. Both treatments given alone (CP, ²²⁴Ra wires) also decreased metastatic burden although less than the combined treatment.

### Example 6

### Single DART Wire Insertion Combined with Gemcitabine Significantly Retarded Pancreatic Carcinoma Growth

The following experiment was performed in order to ascertain the effect of a combined treatment of a single ²²⁴Ra wire and the chemotherapeutic drug gemcitabine (Gemzar).

Group of mice receiving the combination was compared with an inert wire and Gemzar treated groups as well as with Gemzar alone. Mice with Panc-02 tumors (5 mm average length) received ²²⁴Ra wire treatment with or without the chemotherapeutic agent. The drug, (Gemzar, 60 mg/kg), was injected i.v. and the animals were monitored for tumor growth.

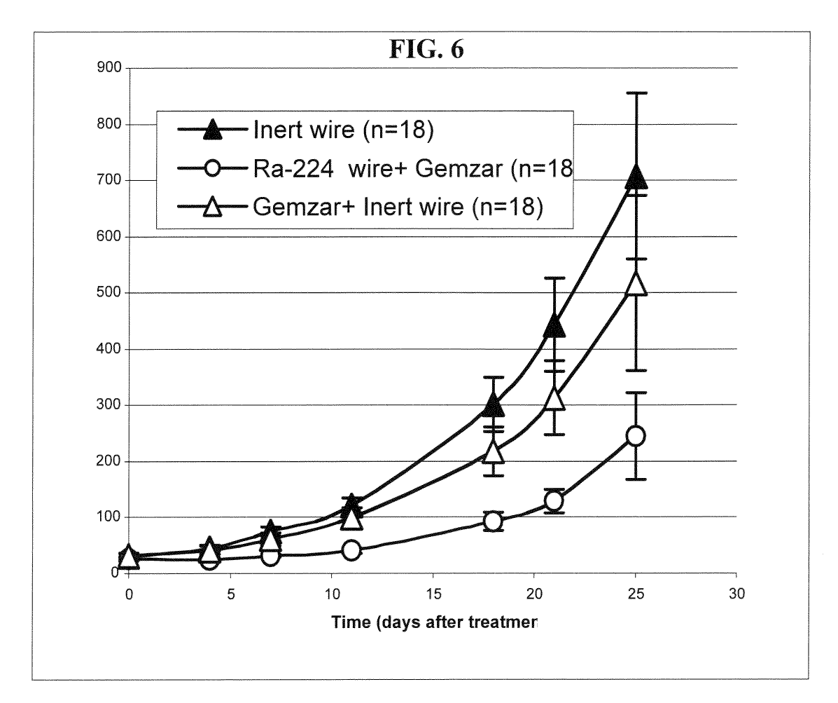

The results presented in FIG. 6 demonstrate that the combined treatment of ²²⁴Ra wire+Gemzar was the most effective modality in local tumor control compared to the effect of Gemzar alone or Inert wire+Gemzar (Pv<0.001) treatment. A significant effect (Pv=0.033) was also seen when comparing the combined treatment with the treatment with ²²⁴Ra wire alone.

### Example 7

### Insertion of Two DART Wires Combined with 5FU Significantly Retarded Colon Carcinoma Tumor Growth and Cured Tumor Bearing Mice

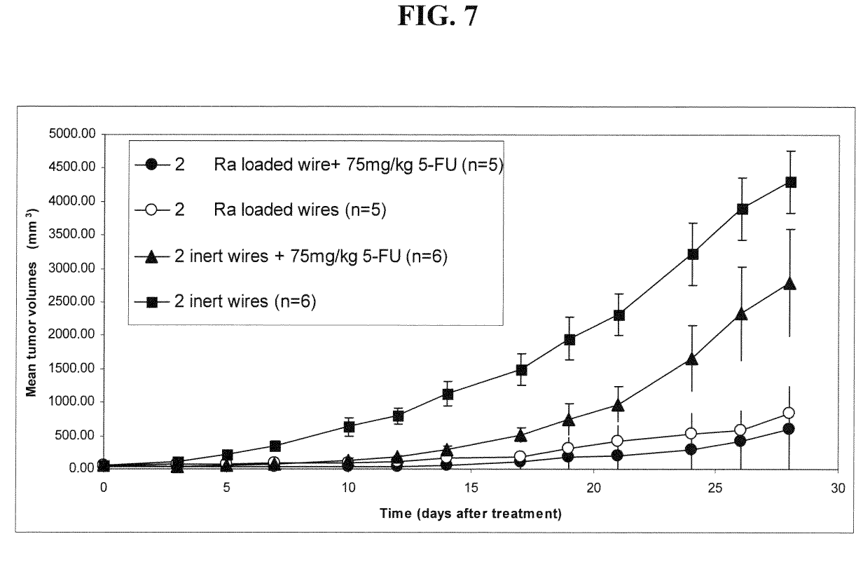

In this experiment, mice were administered 75 mg/kg 5-FU 24 hours prior to treatment with 2 ²²⁴Ra wires. The results presented in FIG. 7A demonstrate that the treatment with two ²²⁴Ra-loaded wires combined with 5-FU had a robust effect on tumor growth retardation and completely cured 4 out of 5 mice (Table 2, herein below). The differences in tumor volumes were significant when compared to the treatment with the ²²⁴Ra wires alone, inert wires or inert with 5-FU (Pv=0.048, 0.005 and 0.039 respectively.

TABLE 2

| | Treatment | | | |
|---|---|---|---|---|
| | | | Two | |
| | | Two | radioactive | Two inert |
| | Two inert | radioactive | wires and 5- | wires and 5- |
| | wires | wires | FU | FU |
| No. of | 6/6 with | 5/5 with tumor | 1/5 with tumor | 6/6 with |
| animals | tumor | | | tumor |
| with tumors | | | | |
| following | | | | |
| treatment | | | | |

### CONCLUSIONS

The above results demonstrate that when squamous cell carcinoma (SCC) cells are treated with 30 μM of Cisplatin for 4 hours, apoptotic cell death mechanisms are initiated. The same happened when cells were DART-exposed to doses higher than 0.8 Gy of alpha particle fluxes. When both treatments were combined according to embodiments of the present invention, enhanced apoptosis was detected. This pattern was also notable when proliferation abilities were tested, as 3 μM of the drug combined with DART alpha irradiation was demonstrated to have a pronounced cytotoxic effect on the cultured cells.

In vivo studies investigated animals bearing SCC tumors treated with a single ²²⁴Ra wire inserted to the center of each SCC tumor accompanied by a regimen of two equal and separated i.v Cisplatin doses (5 mg/kg each) given prior to (one day) and following (4 days) the DART wire insertion. The results indicated that this combination produced a gain when compared to the chemotherapy or the radiotherapy administrated alone.

Combining the positioning of two ²²⁴Ra wires at the tumor base with chemotherapy revealed that the treatment of two intratumoral DART wires associated with two doses of cisplatin caused extensive SCC tumor retardation almost in all treated mice. This method of treatment also resulted in prolongation of the average life expectancy of this group of mice compared to all other treatments.

The findings regarding the conjugation of the DART methodology and cisplatin for the treatment of SCC tumors opened the way for additional tumor models as well as different drugs.

The efficacy of DART against pancreatic tumors was significantly enhanced by the concomitant use of i.v Gemcitabine. Another prominent example is colon carcinoma in which complete tumor eradication was achieved when 5FU was added to treatment with two ²²⁴Ra wires.

Since the combination of DART and cisplatin was observed to enable a major increase in life expectancy of SCC (SQ2 cell line) bearing mice, it was postulated that an inhibition of the metastatic process exists, in light of the fact that BALB/c mice bearing SQ2 derived tumors die primarily from lung metastases. Therefore the present inventors examined the metastatic burden in the lungs of the untreated and treated mice, and found that ²²⁴Ra wires+CP treatment group resulted in a significant reduction of 51% in the lung metastatic load compared to those of the animals that were treated only with wires free of alpha emitters.

To conclude, the results evolving from the experiments presented here indicate that Diffusing Alpha-emitting Radiation Therapy when coupled with chemotherapy, against various solid tumors can produce a synergistic effect inhibiting malignant progress.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of treating a tumor of a subject, the method comprising administering to the subject a therapeutically effective amount of alpha particles and a chemotherapeutic agent, wherein said alpha particles are administered by positioning a non-stable alpha-emitting radionuclide in proximity to and/or within the tumor, so as to administer a dose of alpha particles into the tumor, wherein the method does not comprise administration of an inhibitor of DNA repair, thereby treating the tumor of the subject.

2. The method of claim 1, wherein the tumor is a solid tumor.

3. The method of claim 1, wherein said non-stable alpha-emitting radionuclide is selected from the group consisting of Radium-223, Radium-224, Radon-219 and Radon-220.

4. The method of claim 1, wherein said positioning of said non-stable alpha-emitting radionuclide is effected by at least one radiotherapy device having a surface whereby said alpha-emitting radionuclide is on or beneath said surface.

5. The method of claim 4, wherein said at least one radiotherapy device comprises a wire.

6. The method of claim 1, wherein said non-stable alpha-emitting radionuclide is comprised in a solution.

7. The method of claim 1, wherein said positioning is effected at the base of the tumor.

8. The method of claim 4, wherein said at least one radiotherapy device comprises two radiotherapy devices.

9. The method of claim 1, wherein said tumor is selected from the group consisting of a squamous cell carcinoma tumor (SCC tumor), a pancreatic carcinoma tumor and a colon carcinoma tumor.

10. The method of claim 1, wherein said chemotherapeutic agent is selected from the group consisting of cisplatin, gemcitabine, is 5-fluorouracil (5FU), taxol and doxorubicin.

11. The method of claim 1, wherein when said tumor is a SCC tumor, said chemotherapeutic agent is cisplatin.

12. The method of claim 1, wherein when said tumor is a pancreatic carcinoma tumor, said chemotherapeutic agent is gemcitabine.

13. The method of claim 1, wherein when said tumor is a colon carcinoma tumor, said chemotherapeutic agent is 5-fluorouracil (5FU).

14. A method of treating a tumor of a subject, the method comprising administering to the subject a therapeutically effective amount of alpha particles and a chemotherapeutic agent, wherein said chemotherapeutic agent is administered systemically, wherein said alpha particles are administered by positioning a non-stable alpha-emitting radionuclide in proximity to and/or within the tumor, so as to administer a dose of alpha particles into the tumor and wherein said chemotherapeutic agent is selected from the group consisting of cisplatin, gemcitabine, 5-fluorouracil (5FU), taxol and doxorubicin, thereby treating the tumor of the subject.

15. The method of claim 14, wherein the tumor is a solid tumor.

16. The method of claim 1, wherein said chemotherapeutic agent is a single chemotherapeutic agent.

17. The method of claim 14, wherein said non-stable alpha-emitting radionuclide is selected from the group consisting of Radium-223, Radium-224, Radon-219 and Radon-220.

18. The method of claim 14, wherein said positioning of said non-stable alpha-emitting radionuclide is effected by at least one radiotherapy device having a surface whereby said alpha-emitting radionuclide is on or beneath said surface.

19. The method of claim 18, wherein said at least one radiotherapy device comprises a wire.

20. The method of claim 14, wherein said non-stable alpha-emitting radionuclide is comprised in a solution.

21. The method of claim 14, wherein said positioning is effected at the base of the tumor.

22. The method of claim 18, wherein said at least one radiotherapy device comprises two radiotherapy devices.

23. The method of claim 14, wherein said tumor is selected from the group consisting of a squamous cell carcinoma tumor (SCC tumor), a pancreatic carcinoma tumor and a colon carcinoma tumor.
